(19) United States
(12) Patent Application Publication
(10) Pub. No.: US 2008/0279961 A1
(43) Pub. Date: Nov. 13, 2008

(54) TELOMERE TARGETING AGENTS AS STEM CELLS DIRECTED TREATMENTS

(76) Inventor: Angelika M. BURGER, Baltimore, MD (US)

Correspondence Address: FULBRIGHT & JAWORSKI, LLP, 1301 MCKINNEY, SUITE 5100, HOUSTON, TX, 77010-3095, US

(21) Appl. No.: 12/118,394

(22) Filed: May 9, 2008

Related U.S. Application Data
(60) Provisional application No. 60/917,398, filed on May 11, 2007.

(51) Int. Cl. A61K 33/36; C12N 5/06; C12Q 1/04; G01N 33/574; A61K 31/4375; A61K 31/435; A61K 31/282; A61K 31/4188; A61K 33/24; A61K 31/351; A61K 31/675; A61K 31/704; A61K 31/427; A61K 31/513; A61K 31/437; A61K 31/4353

(52) U.S. Cl. 424/623; 435/375; 435/34; 435/7.23; 514/280; 514/297; 514/365; 514/274; 514/285; 514/284; 424/624; 514/34; 514/283; 514/492; 514/90; 514/393; 514/459; 424/649

(57) ABSTRACT

It is demonstrated in the present invention that G-quadruplex ligands can be used to both shorten telomeres and inhibit telomerase by causing telomere uncapping. The invention relates to compositions and methods of treating cancer stem cells comprising the administration of G-quadruplex ligands, such as 3,11-difluoro-6,8,13-trimethyl-8H-quino[4,3,2-kl]acridinium methosulfate (RHPS4), which can effectively inhibit or reduce the growth of cancer stem cells. The invention also relates to a synergistic effect in inhibiting or reducing the growth cancer stem cells when a G-quadruplex ligand is combined with a mitotic spindle poison, such as paclitaxel, or other agents used in the treatment of cancer and disease. The invention also relates to RHPS4 inducing non-cancerous cell and non-cancerous stem cell proliferation.

This application claims priority to Provisional Application No. 60/917,398, which was filed on May 11, 2007, and which is incorporated by reference herein in its entirety.

### TECHNICAL FIELD

The present invention generally concerns at least the fields of cell biology, molecular biology, cancer biology, and medicine.

### BACKGROUND OF THE INVENTION

Protection of chromosome termini from end-to-end fusion, recombination and degradation is achieved by the telomeres (Blackburn 1991, Blasco 2004). A current model proposes that telomeres form “a cap” at the end of chromosomes. The structure adopted by the G-rich 3′-end overhang is thought to involve a G-quadruplex (Williamson, 1994; Parkinson et al., 2002) and/or loops after invading the double stranded region of the telomere (Griffith et al., 1999). The physical integrity of the telomere “cap” must be intact to allow cell division to proceed (Blackburn 2000). Regulated uncapping occurs normally in dividing cells with the crucial property that a functional telomere rapidly switches back to a capped state (Smith & Blackburn, 1999; Blackburn, 2001). The “uncapping” signal for growth arrest, which is triggered when telomere-mediated chromosome end-protection becomes insufficient due to reduction in telomere length and/or damage to telomere structure, has been recently elucidated. It activates the double strand break (DSB) mediated DNA damage response pathway, because a short, dysfunctional telomere can resemble a double-strand DNA break (Blackburn, 2000; d'Adda di Fagagna et al., 2003; IJpma & Greider, 2003).

In normal somatic cells, which have a finite replicative lifespan, telomeres progressively shorten with successive cell divisions due to the inability of DNA polymerase to replicate DNA fully to the chromosomal end (Makarov et al., 1997; Hayflick and Moorhead, 1961). Cells with self-renewal capacity however, such as stem and cancer cells, possess a telomere maintenance mechanism, namely the expression of the telomere-elongating enzyme telomerase, conferring their immortality. The activation of telomerase has also been shown as an early, crucial event in the genesis of tumor from normal cells and is considered a hallmark of cancer (Kim et al., 1994; Hahn et al., 1999; Hanahan and Weinberg, 2000). Recently it has become evident that telomerase stabilizes telomeres independently of its elongation role through an additional “capping” function and appears to mediate cell survival in the presence of various cytotoxic stresses (Masutomi et al., 2003; Blasco, 2002; Sung et al., 2005).

Since most normal cells lack telomerase and because marked differences exist in telomere length between telomerase-positive adult stem cells or germ cells (average telomere length ˜15 kb) and cancer cells (˜5 kb), inhibiting telomerase activity and/or interfering with the telomere capping function have arisen as attractive targets for cancer treatment (Burger, 1999; Kelland, 2005; Burger, 2007).

Cancer stem cells sustain tumor growth. It is believed that they are responsible for treatment failure and disease recurrence. However, cancer stem cell directed therapeutics do not currently exist. Cancer stem cell characteristics, such as proliferative quiescence (residing in a niche), and the expression of drug efflux pumps as a means of self-protection, make them difficult to treat by conventional anti-cancer agents. Current anti-cancer drugs can only kill bulk tumor cells, and cancer stem cells remain unaffected and can repopulate the tumor leading to disease relapse. Telomerase is an enzyme that is essential for limitless proliferative capacity and immortality consistent with self-renewal properties of stem cells. Cancer stem cells show increased telomerase activity and shorter telomeres. This opens a window for exploiting the telomere-/telomerase complex as a cancer stem cell specific therapeutic target. The present invention provides a solution for the long-felt need found in the art.

### SUMMARY OF THE INVENTION

In particular embodiments, the invention is directed towards methods of cancer treatment and/or prevention. An object of the present invention is to shorten telomeres and/or to cause telomerase inhibition by the use of telomere G-quadruplex ligands in a cancer cell and/or a cancer stem cell. The sequestering of the telomere in a G-quadruplex structure inhibits the catalytic lengthening activity of telomerase, which requires the 3′-end to be in a non-folded form (Zahler et al., 1991). G-quadruplex structures are readily bound and stabilized by small molecule ligands, such as RHPS4 (3,11-difluoro-6,8,13-trimethyl-8H-quino[4,3,2-kl]acridinium methosulfate, a pentacyclic salt, NSC 714187, FIG. 1A) (Gowan et al. 2001) and other G-quadruplex ligands (Reed et al., 2006; Tahara et al., 2006; Burger et al., 2005).

It is also an object of the present invention to inhibit growth of cancer stem cells by RHPS4. It is a further object of the present invention to inhibit cancer stem cells by RHPS4. Sensitivity to growth inhibition by RHPS4 appears correlated to telomere length as shown in a panel of human tumor lines that were grown in the clonogenic assay, also known as the human tumor stem cell assay (Hamburger & Salmon, 1977; Cookson et al., 2005). Similarly, in vivo treated UXF1138L xenograft tissue had a decreased clonogenicity and exhibited mitotic abnormalities, consistent with telomere dysfunction.

Another object of the present invention is to provide a synergistic combination of RHPS4 and an additional cancer therapy to inhibit growth of cancer cells and/or cancer stem cells, although in an alternate embodiment it is an additive combination. It has been demonstrated that the telomere targeting agent RHPS4 and the exemplary tumor “debulking” agent paclitaxel act in a synergistic manner and can cause complete remission of UXF1138L xenografts, for example. Also shown herein, the G-quadruplex ligand RHPS4 can selectively eradicate cancer stem cells over normal adult stem cells. Also described is the synergy, above what one of skill in the art would expect, of RHPS4 with other anti-cancer agents.

One embodiment of the invention is a method to inhibit the growth of a cancer stem cell, comprising contacting the cancer stem cell with an effective amount of G-quadruplex ligand. In specific embodiments of the invention, the G-quadruplex ligand comprises 3,11-difluoro-6,8,13-trimethyl-8H-quino[4,3,2-kl]acridinium methosulfate (RHPS4), BRACO-19, telomestatin, or a functionally active derivative thereof. In another embodiment, the invention further comprises contacting the cancer stem cell with an additional anti-cancer drug. In specific embodiments of the invention the additional anti-cancer drug reacts additively or synergistically with the G-quadruplex ligand. In another embodiment of the invention, the additional anti-cancer drug is gemcitabine, a mitotic spindle poison, a heat shock protein inhibitor, an anti-metabolite, a cross-linking agent, a platinum compound, or a double strand break (DSB)-inducing agent. In another specific embodiment of the invention the anti-cancer agent comprises Paclitaxel, 17-AAG, doxorubicin, cisplatin, carboplatin, arsenic trioxide, Vincristin, Vinblastion, Vinflunine, docetaxel, epithiolones or sodium meta arsenite.

Another embodiment of the invention is a method of treating or lessening the severity of cancer in a mammal in need of such treatment by inhibiting the growth of a cancer stem cell, comprising administering a therapeutically effective amount of a G-quadruplex ligand to the mammal. Another embodiment of the invention is a method of inducing non-cancerous cell proliferation in a mammal. In a specific embodiment, the method concerns including non-cancerous stem cell proliferation in a mammal.

Another embodiment of the invention is drawn to a method of treating metastatic cancer or cancer resistant to standard chemotherapy. Recently, stem cells have been implicated in certain cancers (see, for example, Hermann et al., 2007). For example, it has been found that human pancreatic cancer tissue contains cancer stem cells defined by CD133 expression that are exclusively tumorigenic and highly resistant to standard chemotherapy. In the invasive front of pancreatic tumors, a distinct subpopulation of CD133(+) CXCR4(+) cancer stem cells was identified that determines the metastatic phenotype of the individual tumor. Depletion of the cancer stem cell pool for these migrating cancer stem cells virtually abrogated the metastatic phenotype of pancreatic tumors without affecting their tumorigenic potential. Therefore, in certain instances a cancer stem cell is essential for metastatic cancer or cancer resistant to a cancer therapy. Therefore, the invention is also drawn to treating metastatic cancer or cancer resistant to a cancer therapy.

An embodiment of the invention is a method of treating cancer in a mammal in need of such treatment comprising administering a therapeutically effective amount of a G-quadruplex ligand to the mammal, wherein the G-quadruplex ligand induces non-cancerous cell proliferation. In a specific embodiment, the G-quadruplex ligand also inhibits cancer cell growth or cancer stem cell growth while also inducing non-cancer cell proliferation or non-cancer stem cell proliferation.

An embodiment of the invention is a method increasing proliferation of a non-cancerous stem cell in an individual, comprising the step of delivering an effective amount of a G-quadruplex ligand to the individual. In a specific embodiment of the invention, the G-quadruplex ligand is 3,11-difluoro-6,8,13-trimethyl-8H-quino[4,3,2-kl]acridinium methosulfate (RHPS4). In another embodiment of the invention is a method of increasing proliferation of a non-cancerous stem in vitro. In a specific embodiment, the method increases proliferation of a non-cancerous stem cell in an individual. In some embodiments, a stem cell that is not a cancer stem cell is referred to as a normal stem cell.

Other and further objects, features and advantages would be apparent and eventually more readily understood by reading the following specification and/or any examples of the present embodiments of the invention are given for the purpose of the disclosure.

### BRIEF DESCRIPTION OF THE DRAWINGS

FIG. 1A shows the structure of RHPS4 (NSC 714187), 3,11-difluoro-6,8,13-trimethyl-8H-quino[4,3,2-kl]acridinium methosulfate. FIG. 1B shows the design of in vivo xenograft studies, wherein fragments (grey spheres) from an untreated donor animal were implanted into recipient mice, which were treated orally with 5 mg/kg/d RHPS4 every 3 days for 8 times after randomization (=6 days after tumor transplantation). Tissue from the vital rim of three tumors from each group was homogenized, digested and primary cultures as well as clonogenic growth assays prepared from single cell suspensions. Primary cultures were used for analysis of telomere length. The control mouse group was always derived from untreated tumor fragments, but from the same initial passage and donor mouse as were the RHPS4 treated tumors. A total of 4 passages were analyzed.

FIG. 2A shows the comparison of the antiproliferative activity of RHPS4 in uterus (UXF1138L) and prostate (PC3) cancer cell lines grown as colonies in the HTCA (broken lines) or as monolayers in the MTT assay (solid lines). UXF1138L # of colonies 100%=56; O.D. 100%=0.7±0.03; PC3 # off colonies 100%=94±18, O.D. 100%=0.75±0.01. FIG. 2B shows that RHPS4 is two log-fold more active in MCF-7 cells grown as colonies (IC50=0.04 microM, grey arrow) in the HTCA than in MCF-7 whole cell populations (IC50=2 microM, grey arrow). # of colonies 100%=101±36, O.D. 100%=2.7±0.19. FIG. 2C shows that the effects of RHPS4 on colonies of HEK293T embryonic kidney cells in the HTCA and human cord blood mononuclear cells in the methylcellulose assay. Colony growth of HEK293T cells is compared to the growth of the bulk cell population by MTT assay. Data are depicted as % of control growth and mean number of colonies per well (HTCA), or the mean optical density measured at 550 nm (MTT assay). All data represent the mean of three independent experiments plus standard deviation. Cord blood # of colonies in the control (100%)=31.6; HEK293T, # of colonies 100%=187.25; O.D. 100%=1.215. Data shown are representative of three independent experiments.

FIG. 3A shows the tumor growth inhibition of subcutaneous UXF1138L xenografts in passage 3 of chronic RHPS4 exposure or vehicle only treated controls. Drug was given orally every three days, 8 times. The median relative tumor volumes are shown in percentage; the tumor size at randomization was set as 100%. FIG. 3B shows the effects of RHPS4 in vivo treatment on tumor colony growth/stem cell formation in vitro from tumors in A. Colony count: Control=84±SD 29.6; RHPS4 5 mg/kg/d=46±SD 5.1. FIG. 3C shows the telomere restriction fragment length (TRF) measured in primary cultures from tumors in FIG. 3A (passage 3, P3) and the previous experiment (passage 2, P2) by Southern blot. Telomeres of treated UXF1138L xenografts (T) were ˜1 kb shorter than control tissues (TRF P2, lane 2: ˜5.7 kb compared to lane 3: ˜4.7 kb; TRF P3, lane 4: ˜4.6 kb versus lane 5: ˜3.4 kb). Lane 1=molecular weight standard supplied with the Roche Telo-TAGGG kit. FIG. 3D-3F shows the loss of nuclear hTERT expression and occurrence of atypical mitotic figures after RHPS4 treatment. Control tissues were probed with mouse IgG (isotype negative control, FIG. 3D), and monoclonal hTERT antibodies (FIG. 3E). RHPS4 treated tissue was stained for hTERT protein expression (FIG. 3F), sections were counterstained with hematoxylin. RHPS4 treatment leads to loss of nuclear hTERT expression (FIG. 3F) and increase in mitotic abnormalities e.g. ring chromosomes (enlargement and black arrows) and anaphase bridges.

FIG. 4A shows the expression of hTERT in UXF1138L cells. Expression of hTERT in UXF1138L cells treated with PBS (control) or 1 microM RHPS4 for 24 hrs. In RHPS4 treated cells nuclear hTERT signal is attenuated. The white arrows in the lower panel to the right indicate distribution of hTERT in the cytoplasm. Cells were dual labeled against hTERT (green) and for DNA (blue). Bars=15 microns. FIG. 4B shows the western blot of nuclear extracts from UXF1138L cells treated for 1, 6, and 24 hours with 1 microM of RHPS4. Membranes were developed with anti-gamma-H2AX antibodies (upper panel), and/or gels directly stained with Coomassie blue (lower panel, equal loading control). FIG. 4C shows gamma-H2AX expression in nuclei of UXF1138L cultured in the absence (top) and presence of RHPS4 (bottom). FIG. 4D shows the enlargement of gamma-H2AX positive, DAPI stained UXF1138L cells from FIG. 3C (indicated by white box) in (FIG. 3D left panel), and UXF1138L interphase nuclei probed with human telomere and centromere paints by FISH in (FIG. 3D right panel). FIG. 4E shows the metaphase spreads from treated (24 h) and control UXF1138L cells. RHPS4 exposure for 24 hrs (1 microM) results in ring and dicentric chromosomes (white arrows) that are responsible for the formation of anaphase bridges.

FIG. 5A shows the combination of RHPS4 and paclitaxel in UXF 1138L cells in vitro is synergistic. Shown are the combination indices against fractional effect (on growth) for the in vitro combination of RHPS4 and paclitaxel at a fixed ratio of their individual IC₅₀ values. CI (combination index) values are given for the doses effecting 50, 75 and 90% growth inhibition compared to control (ED₅₀, ED₇₅ and ED₉₀ respectively); CI values below 1 indicate synergistic drug effects (Chou and Talalay, 1984). FIG. 5B shows the tumor growth inhibition of UXF1138L xenografts by Paclitaxel given at 20 mg/kg i.v. on days 1, 15. Shown is the median relative tumor volume in percentage. Control and RHPS4 groups had to be sacrificed on day 21, while the combination showed complete remissions and was terminated after 40 days. Minor remissions were seen on days 7-10 (n=6 mice). The combination of RHPS4 (5 mg/kg p.o. twice weekly) and paclitaxel (single dose 20 mg/kg i.v. on day 1) was highly effective and did lead to complete, durable remissions of UXF1138L xenografts. RHPS4 alone produced only marginal growth inhibition (n=5 mice). FIG. 5C shows the box plots for atypical mitosis in UXF1138L tumors. Residual tissues masses from RHPS4/Paclitaxel treated tumors show pronounced induction of atypical mitoses compared to vehicle control. The number of mitotic abnormalities is further increased in the combination group from that seen with single agent RHPS4. Control=0.35±0.07, RHPS4 alone=1.25±0.19, RHPS4+Paclitaxel=1.8±0.08. The line within the box marks the median, whiskers indicate the 10^{th} and 90^{th} percentiles of the box plots.

FIG. 6 demonstrates the need for targeting cancer stem cells. Conventional chemotherapy or radiation (upper pathway), for example, allows repopulation of the cancer cells while cancer stem cell targeting treatments such as discussed herein will create a durable remission and/or cure. It is a schematic overview of the role of cancer stem cells in the response of bulk tumor populations to conventional chemotherapy and disease relapse (A). Consequences of adding cancer stem cell-directed therapies to conventional debulking agents (B). Bulk tumor populations are depicted as actively cycling cells. Cancer stem cells are presented as long-term stem cells (LT-CSC) that are proliferative quiescent (in G0 phase) and reside in a niche environment, and as short-term cancer stem cells (ST-CSC) that are actively cycling and transiently amplify. Cancer stem cells express a high density of drug efflux pumps such as BCRP (small dark circles). Tumor relapse results from reconstitution of the bulk cell population from cancer stem cells. As a result bulk cells also express drug efflux pumps and render resistant to conventional cytotoxics that are substrates of the pumps. The insert on the right hand corner shows telomeres and telomerase as cancer stem cell targets and an example of a target within the self-renewal and differentiation pathways. HSC, hematopoietic stem cells are contrasted for telomere length and telomerase activity expression relative to LSCs, leukemic stem cells.

FIG. 7 shows that cancer stem cells have short telomeres. FIG. 7A shows the side population in three prostate cancer cell lines. FIG. 7B shows the telomere content of the prostate cancer cell lines in 7A as determined by Southern blotting. Telomere content is a surrogate for telomere length (Fordyce et al., 2005). FIG. 7C shows the correlation between telomere length and chemosensitivity to RHPS4. Mean TRF values and IC50 values were ranked for available comparisons, Spearman rank analyses were performed, r=0.75 (r=correlation coefficient). Because of the wide range of actual IC₅₀ values, the correlation analysis had to be performed using the Spearman rank statistics. The Spearman rank correlation coefficient is also a better indicator that a relationship exists between two variables when the relationship is nonlinear. The data are presented as a scatter plot with regression line.

FIG. 8A-C demonstrates that tumors with shorter telomeres are more sensitive to RHPS4. FIG. 8A shows a sensitivity profile of 36 human tumor tissues grown in the human tumor stem cell assay (soft agar clonogenic assay). In the semisolid soft agar matrix, only cells capable of anchorage independent growth, a characteristic of stem cells, can grow. Bars to the left on FIG. 9A are sensitive tumors, while bars to the right are more resistant tumor types. Arrows indicate the most sensitive tumors, the prostate cancer PC-3 and the uterus carcinoma UXF 1138 and also connect the growth data with the Southern blot telomere length results in figure FIG. 9C (lanes 4 and 5). FIG. 9B shows four cell lines, two with very long telomeres Saos-2 (14 kb) and HEK293T (16 kb) and two with very short telomeres UXF 1138 (2.7 kb) and H460 (4 kb) grown as bulk cells in a “standard” 96-well plate MTT assay and treated with the telomere targeting agent RHPS4. FIG. 8C shows a southern blot detection of mean telomere length in human tumor cell lines and xenografts. The arrow in lane 2 indicates residual mouse telomere signal from xenograft primary culture, which contained mouse fibroblasts. The mean TRF length of the human telomere signal was determined relative to a molecular weight standard and taken as the mean of the high-density telomere smear (e.g., indicated in lane 5 as a horizontal line.). MWM, molecular weight marker; lane 1, low TRF standard; lane 2, LXFA 289; lane 3 RXF 393; lane 4, OVXF 899; lane 5, UXF 1138; lane 6, LXFL 529; lane 7, DU145.

FIG. 9 shows that RHPS4 induces the growth of normal stem cells at low concentrations and this is associated with an induction of cytokines. FIG. 9A shows that RHPS4 induces the growth of normal monkey bone marrow stem cells grown in methylcellulose at low drug concentrations that kill cancer stem cells. FIG. 9B shows the secretion of stem cell associated cytokines after RHPS4 treatment into the supernatant. On the left, the breast cancer cell line MCF-7 exhibits an inhibition of tumor necrosis factor alpha, VEGF, GMCSF and GCSF, whereas on the right the normal embryonic kidney fibrobast line HEK293T shows and induction in cytokine secretion, particularly VEGF. Hence the data demonstrates that RHPS4 inhibits the secretion of stem cells associated cytokines by breast tumor stem cells, but induces cytokine secretion in normal stem cells.

FIG. 10 provides an embodiment for the method of telomerase inhibition and telomere targeting (left). On the right, the mechanism of telomere targeting and the action of a G-quadruplex ligand on the 3′ end overhang are shown. A. Depicts a tumor cell with an intact telomere/telomerase and associated protein complex in the nucleus. B. Shows the distortion of the telomere/telomerase complex after the addition of a G-quadruplex ligand to a tumor cells and events following the stabilization of the G-quadruplex. HTERT and Pot1 are translocated into the cytoplasm and hTERT is being degraded in the ubiquitin-proteasome system, hence telomerase activity is inhibited as an effect of telomeretargeting. In addition, DNA-damage signaling is induced.

### DETAILED DESCRIPTION OF THE INVENTION

In keeping with long-standing patent law convention, the words “a” and “an” when used in the present specification in concert with the word comprising, including the claims, denote “one or more.” Some embodiments of the invention may consist of or consist essentially of one or more elements, method steps, and/or methods of the invention. It is contemplated that any method or composition described herein can be implemented with respect to any other method or composition described herein.

As used herein, the term “therapeutically effective amount” refers to an amount that results in an improvement or remediation of at least one symptom of the disease or condition. Those of skill in the art understand that the effective amount may improve the patient's or subject's condition, but may or may not be a complete eradication of a symptom or cure of the disease and/or condition.

The term “G-quadruplex” as used herein refers to nucleic acid sequences rich in guanine that can form a square arrangement with four strands of nucleic acids. One guanine from each of the four nucleic acid strands hydrogen bonds the other three guanines. In one embodiment of the invention, a G-quadruplex is stabilized by the addition of a G-quadruplex ligand. In a further embodiment of the invention, G-quadruplex formation disrupts telomere maintenance.

The term “G-quadruplex ligand” as used herein refers to a structure, such as, for example, a small molecule, inorganic salt, peptide, or protein which stabilizes, or otherwise modulates the formation of the G-quadruplex structure, especially those formed by human telomeric DNA. In one embodiment of the invention, the ligand is RHPS4 and other pentacyclic acridinium G-quadruplex ligands (see, for example, U.S. Pat. No. 7,115,619; Phatak et al., 2007), BRACO-19, quinoline-substituted triazines, such as, for example, 115405 and 12459 (see, for example, Riou et al., 2002), telomestatin and other natural G-quadruplex ligands (see, for example, Shammas et al., 2004; Kim et al., 2002), a porphyrin G-quadruplex ligand, such as, for example, TMPyP4 [tetra(N-methyl-4-pyridyl)-porphyrin chloride (see, for example, Shammas et al., 2003), 2,6-bis[3-(N-Piperidino)propionamido]anthracene-9,10-dione (PPA) (see, for example, Shammas et al., 2004), or derivatives of any of the foregoing. In one embodiment of the invention, G-quadruplex ligands lead to senescence in cancer stem cells. Applicant notes that the invention is not drawn to any particular G-quadruplex ligand, and that the invention encompasses any G-quadruplex ligand known to one of ordinary skill in the art, readily identifiable by one of ordinary skill in the art, as well as any after-arising G-quadruplex ligand. In another embodiment, the G-quadruplex ligand induces cell proliferation.

“Cancer stem cell” as used herein refers to a subpopulation of cancer cells. For example, when a single cancer stem cell from this subpopulation is transplanted, one cancer stem cell can regenerate another population of cancer cells (tumorigenic cancer cells). In one embodiment of the invention, a cancer stem cell has the ability of self-renewal. In a further embodiment of the invention, a cancer stem cell has the ability to differentiate into multiple cell types.

The term “non-cancerous stem cell” as used herein refers to a subpopulation of non-cancerous cells. For example, when a single stem cell from this subpopulation is transplanted, the stem cell can regenerate the complete tissue from which it was derived. In one embodiment of the invention, a stem cell has the ability to self-renew. In a further embodiment of the invention, a non-cancerous stem cell has the ability to differentiate into multiple cell types. In a specific embodiment of the invention, the non-cancerous stem cell is a hematopoietic stem cell, a kidney stem cell, an embryonic stem cell, an adult stem cell, a muscle stem cell, a brain stem cell, a liver stem cell, a skin stem cell.

The term “mitotic spindle poison” as used herein refers to a molecule such as a small molecule, peptide or protein that interrupts the formation of spindles during cell division. In one embodiment of the invention, the mitotic spindle poison is Paclitaxel, Vincristin, Vinblastion, Vinflunine, docetaxel, or epithiolones.

The term “synergistic” or “synergistically” as used herein refers to the addition of two reactants which may or may not react in the same pathway with each other, from which the resulting product of the reaction proceeds to a further extent than one of skill in the art would predict. In a specific embodiment, two compounds act synergistically when the result achieved upon using them in combination is greater than the sum of the results of the compounds when used separately.

The term “additive” or “additively” as used herein refers to the addition of two reactants which may or may not react in the same pathway with each other, from which the resulting product of the reaction proceeds as expected by the addition of the two reactants added separately. In a specific embodiment, two compounds act additively when the result achieved upon using them in combination is about equivalent to the sum of the results of the compounds when used separately.

The phrase “effective amount” as used herein means that amount of a compound, material, or composition comprising a compound of the present invention that is effective for producing some desired effect, e.g., halting the growth of, reducing the size of, and/or causing apoptosis in a cancer stem cells. In one embodiment, the effective amount is enough to reduce or eliminate at least one cell. One of skill in the art recognizes that an amount may be considered effective even if the cancer stem cell is not totally eradicated but decreased partially. For example, the spread of the cancer may be halted or reduced, a side effect from the cancer may be partially reduced or completed eliminated, and so forth. In another embodiment of the invention, the effective amount increases the proliferation of at least one non-cancerous cells. In a specific embodiment of the invention the proliferating non-cancerous cell is one or more non-cancerous stem cells. In another embodiment of the invention, the effective amount inhibits the growth of at least one cancer cell. In a specific embodiment of the invention, the cancer cell is a cancer stem cell.

The term “derivative” as used herein is a compound that is formed from a similar compound or a compound that can be considered to arise from another compound, if one atom is replaced with another atom or group of atoms. Derivative can also refer to compounds that at least theoretically can be formed from the precursor compound.

The terms “functionally active derivative” or “functional derivative” is a derivative as previously defined that retains the function of the compound from which it is derived.

The terms “inhibit,” “inhibitory,” or “inhibitor” as used herein refers to one or more molecules that interfere at least in part with the growth or activity of the molecule or cell it inhibits. The inhibition of a stem cell may be the inhibition of growth of at least one cell. In one embodiment of the invention, G-quadruplex ligands inhibit the growth of cancer stem cells. In another embodiment of the invention, small molecules inhibit the activity of specific proteins or pathways.

As used herein, the term “proliferate” and all of its forms and tenses refer to the growth or division of one more cells. In one embodiment of the invention, a G-quadruplex induces the proliferation of non-cancerous cells.

The term “preventing” as used herein refers to minimizing, reducing, suppressing the risk of developing, or delaying the onset of a disease state (such as cancer) or parameters relating to the disease state or progression or other abnormal or deleterious conditions.

As used herein, “treat” and all its forms and tenses (including, for example, treat, treating, treated, and treatment) refer to both therapeutic treatment and prophylactic or preventative treatment. Those in need thereof of treatment include those already with a pathological condition of the invention (including, for example, a cancer) as well as those in which a pathological condition of the invention is to be prevented. In certain embodiments, the terms “treating” and “treatment” as used herein refer to administering to a subject a therapeutically effective amount of a composition so that the subject has an improvement in the disease or condition. The improvement is any observable or measurable improvement. Thus, one of skill in the art realizes that a treatment may improve the patient's condition, but may not be a complete cure of the disease. Treating may also comprise treating subjects at risk of developing a disease and/or condition of the invention.

As used herein the term “metastatic” (and all other forms and tenses, including, for example, metastasis, metastasize, etc.) when used alone or in conjunction with cancer refers to the spread of a cancer from one part of the body to another, unless otherwise indicated by the use or context. Typically, a tumor formed by cells that have spread is called a “metastatic tumor” or a “metastasis.” The metastatic tumor contains cells that are like those in the original (primary) tumor.

As used herein “resistant” (and all other forms and tenses, including, for example, resistance, etc.) when used alone or in conjunction with cancer means a cancer that does not respond to an a cancer therapy, unless otherwise indicated by the use or context. The cancer may be resistant at the beginning of therapy, or it may become resistant during therapy. The invention also encompasses a refractory cancer.

One embodiment of the invention is a method of inhibiting the growth of a cancer stem cell comprising contacting said cancer stem cell with an effective amount of G-quadruplex ligand. In a specific embodiment of the invention the G-quadruplex ligand comprises 3,11-difluoro-6,8,13-trimethyl-8H-quino[4,3,2-kl]acridinium methosulfate (RHPS4), BRACO-19, telomestatin, or a functionally active derivative thereof. Functionally active derivatives of RHPS4, telomestatin or BRACO-19 would be known to one of skill in the art or routine to screen for. Additional derivatives of RHPS4 have also been defined in U.S. Pat. No. 7,115,619 incorporated by reference here in full.

In another embodiment of the invention, the method further comprises an additional anti-cancer drug. In a further embodiment, the anti-cancer drug is selected from the group consisting of a mitotic spindle poison, a heat shock protein inhibitor, an anti-metabolite, a cross-linking agent, a platinum compound, a HDAC inhibitor, a DSB-inducing agent, a arsenical, a Poly(ADP-Ribose) polymerase (PARP) inhibitor, an hTR, hTERT or dyskerin antisense compound or siRNA. In specific embodiments, the anti-cancer drug comprises Paclitaxel, 17-AAG, cisplatin, doxorubicin, sodium meta arsenite, carboplatin, oxaliplatin, docetaxel, 17-DMAG, gemcitabine, sodium butyrate, SAHA, paclitaxel, Vincristin, Vinblastion, Vinflunine, docetaxel, or epithiolones. In a further embodiment of the invention, the additional anti-cancer drug reacts additively or synergistically with the G-quadruplex ligand.

One embodiment of the invention is a method of treating or lessening the severity of cancer in a mammal in need of such treatment by inhibiting the growth of a cancer stem cell comprising administering a therapeutically effective amount of a G-quadruplex ligand to the mammal. In another embodiment of the invention, the G-quadruplex ligand increases non-cancerous cell proliferation. In a specific embodiment, the proliferating cells are non-cancerous stem cells.

An embodiment of the invention is a method of treating cancer in a mammal in need of such treatment comprising administering a therapeutically effective amount of a G-quadruplex ligand to the mammal, wherein the G-quadruplex ligand induces non-cancerous cell proliferation and inhibits the growth of cancer cells. In a specific embodiment of the invention, the cancer cells are cancer stem cells. In another specific embodiment of the invention, the non-cancer cells are non-cancer stem cells. In a further embodiment of the invention, the G-quadruplex ligand comprises 3,11-difluoro-6,8,13-trimethyl-8H-quino[4,3,2-kl]acridinium methosulfate (RHPS4). In a specific embodiment of the invention, the concentration of the G-quadruplex ligand is between 0.01 micromolar and 1 micromolar.

One embodiment of the invention is a method inducing one or more non-cancer cells to proliferate, self-renew, divide, or differentiate. In a further embodiment of the invention, the non-cancer cell is an adult or embryonic stem cell. In another specific embodiment of the invention, the G-quadruplex ligand induces one more or more non-cancer cells to proliferate, self-renew, divide or differentiate. In a further embodiment of the invention, the G-quadruplex ligand is the compound 3,11-difluoro-6,8,13-trimethyl-8H-quino[4,3,2-kl]acridinium methosulfate (RHPS4). In another embodiment of the invention, the G-quadruplex ligand also inhibits the growth of one or more cancer cells. In a specific embodiment of the invention, the cancer cell is a cancer stem cell. In another embodiment, the G-quadruplex ligand also induces the proliferation of non-cancer cells. In a specific embodiment, the non-cancer cell is a normal, adult, or embryonic stem cell. One of skill in the art will know of techniques, assays, and methods to distinguish cancer cells from non-cancerous cells, for example, by performing a biopsy.

The present invention generally concerns compositions and methods useful in the treatment of cancer in an individual. In particular aspects of the invention, the invention generally concerns compositions and methods for telomere uncapping in a cancer cell in an individual that has cancer or that is suspected of having cancer. The individual may be of any kind including a mammal, but in particular aspects the individual is a human, dog, cat, or horse, for example.

I. Cancer Stem Cells

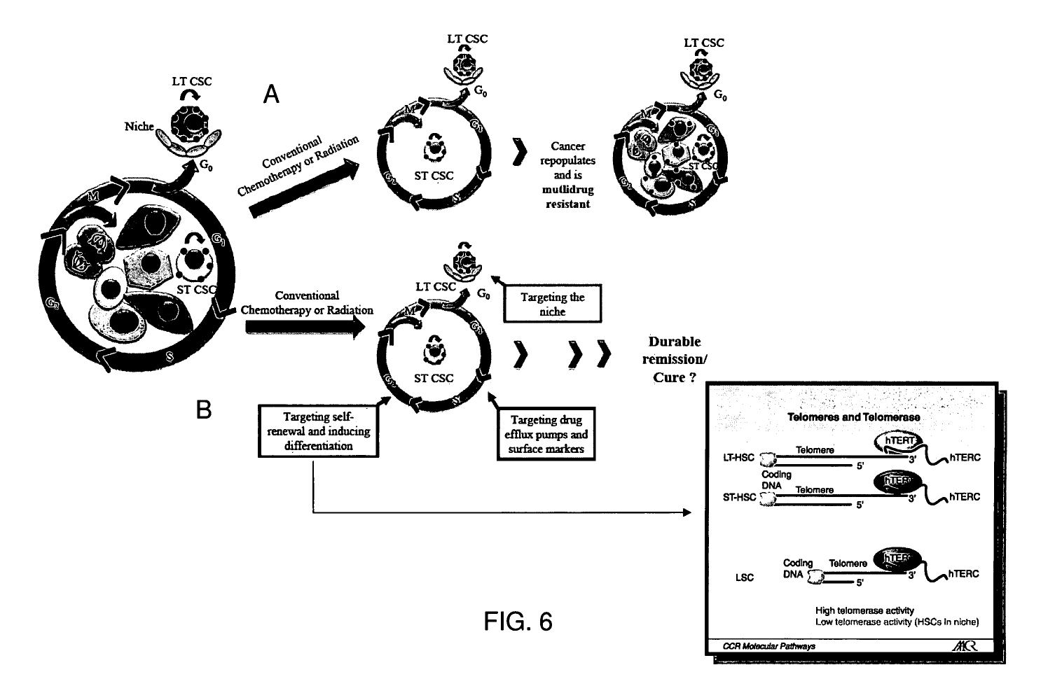

In a particular embodiment of the invention, the cancer that is treated comprises cancer stem cells. Currently, cancer stem cells have been defined as “a small subset of cancer cells within a cancer that constitute a reservoir of self-sustaining cells with the exclusive ability to self-renew and to cause the heterogeneous lineages of cancer cells that comprise the tumor” (Clarke et al. 2006; Hill et al. 2007). While a normal stem cell is considered to be multipotent or capable of differentiating into different cell phenotypes across all lineages, the definition of a cancer stem cell does not include multipotency. Normal stem cells also require specific environments (stem cell niche) comprising other cells, stroma and growth factors for their survival (Blanpain et al. 2004). Limitless proliferative potential (self-renewal), self-protection (expression of drug efflux pumps), and proliferative quiescence (G₀ arrest) are general stem cell properties. An intriguing property of normal and cancer stem cells are the high levels of expression of the drug efflux pumps P-glycoprotein (Pgp/ABCB1) and BCRP (breast cancer resistance protein, ABCG2) (FIG. 6) (Donnenberg and Donnenberg, 2005; Chumsri et al. 2007). While ABC transporters provide a mechanism of self-protection to normal stem cells, in cancer stem cells they confer multidrug resistance against most of the clinically used standard cytotoxic agents that are substrates of the pumps. This stem cell characteristic allows for the isolation of a stem cell enriched cell fraction termed the side population. Goodell and colleagues discovered that the simultaneous display of Hoechst fluorescence at two emission wavelengths revealed a small and distinct subset of whole bone marrow cells that had phenotypic markers of multipotential hematopoietic stem cells (HSC) (Goodell et al., 1996).

Cancers comprising cancer stem cells can be defined, identified or distinguished through cell surface markers that are specific for a disease (Table 1). Although causal relationships between a particular surface phenotype and stem cell function have not been established, surface markers offer attractive cancer stem cell targets. Specifically, monoclonal antibodies could be employed to identify and isolate stem cells. Other ways to identify cancer stem cells include their potential of anchorage-independent growth in semisolid matrices such as soft agar or methylcellulose, or as floating spheroids without any solid support (Fiebig et al., 2004; Locke et al., 2005; Ogawa et al., 1976).

Importantly, agents can be used to attack quiescent cells such as “long-term” stem cells homing in niches (FIG. 6). Breast, colon, and prostate cancers share the CD44 epitope (Table 1). Bivatuzumab should be used in adjuvant settings to prevent relapse and/or eradicate breast, colon or prostate cancer stem cells. Table 1 provides a list of known cancer stem cells and specific markers as well as potential treatments. One of skill in the art realizes that the markers in table 1 are exemplary, and that one of skill in the art would know of other such markers to identify cancer stem cells.

TABLE 1

| | | Stem Cell Target | |
|---|---|---|---|
| Tumor Type | Stem Cell Surface Marker | Disease Specific | Agents |
| Chronic myelogenous | CD34⁺CD38⁻ | BCR-ABL | BMS-214662 |
| leukemia | | | |
| Acute myeloid | CD34⁺CD38⁻ | CD33 | Gemtuzumab ozogamicin |
| leukemia | | CD44 | (Mylotarg) |
| | | | CD44 antibody |
| | | | bivatuzumab |
| Acute lymphoid | CD34⁺CD38⁻ | NE | NE |
| leukemia | | | |
| Multiple myeloma | CD138⁻CD34⁻ | CD20 | Rituximab |
| Breast cancer | CD44⁺/CD24^{-/low} | CD44 | CD44 antibody |
| | | Let-7 | Let-7 mimics |
| | ALDH1 | Aldehyde | Disulfiram |
| | | dehydrogenase 1 | |
| | | (ALDH1) | |
| Brain cancer | CD133⁺/Nestin⁺ | VEGF (niche) | VEGF antibody |
| | population | | bevacizumab |
| Prostate cancer | CD44⁺/α₂β₁^{hi}/CD133⁺ | CD44 | CD44 antibody |
| | | | bivatuzumab |
| Lung cancer | Side population | ABC Transporters | Zosuquidar, Valspodar |
| Colon caner | CD133⁺ | B-Catenin | Wnt inhibitors |
| | | AKT | AKT inhibitors |
| Head and neck cancer | CD44⁺ | CD44 | CD44 antibody |
| | | EGFR | bivatuzumab |
| | | | Cetuximab, Pantitumumab, |
| | | | Erlotinib |
| Pancreatic cancer | CD44⁺CD24⁺ESA⁺ | CD44 | CD44 antibody |
| | | Sonic hedgehog | bivatuzumab |
| | | | Cyclopamine |

In a particular embodiment of the invention, the composition comprises at least one compound that causes telomere uncapping in a cancer cell. In specific embodiments of the invention, the composition comprises a G-quadruplex ligand. In a specific embodiment, the composition comprises the compound 3,11-difluoro-6,8,13-trimethyl-8H-quino[4,3,2-kl]acridinium methosulfate (RHPS4). In another specific embodiment, the composition comprises the compound BRACO-19. In another specific embodiment, the composition comprises derivatives of RHPS4, telomestatin or BRACO-19.

In particular embodiments of the invention, the composition further comprises at least one compound that causes reduction in telomere length in a cancer cell. In a specific embodiment of the invention comprises an additional anti-cancer agent. In specific embodiments of the invention, the composition comprises a platinum compound. In a specific embodiment, the composition comprises cisplatin.

In a particular embodiment of the invention, the composition comprises at least a first and a second compound that act synergistically to target telomeres in a cancer cell. In specific embodiments of the invention, a first compound is the compound 3,11-difluoro-6,8,13-trimethyl-8H-quino[4,3,2-kl]acridinium methosulfate (RHPS4). In a further embodiment of the invention, the second compound is an anti-cancer agent. In specific embodiments of the invention, said second compound is a platinum compound. In further specific embodiments, a second compound is cisplatin. In another specific embodiment, a second compound is carboplatin.

In other embodiments of the invention, an individual that has cancer comprising a cancer stem cell or that is suspected of having cancer comprising a cancer stem cell is administered a composition of the invention. The composition may be administered to the individual in any suitable manner, but in specific embodiments the drug is administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

In a particular embodiment of the invention, the composition that is administered to an individual that has cancer comprising a cancer stem cell or that is suspected of having cancer comprising a cancer stem cell comprises at least one compound that causes telomere uncapping in a cancer stem cell. In specific embodiments of the invention, the composition that is administered to an individual that has cancer comprising a cancer stem cell or that is suspected of having cancer comprising a cancer stem cell comprises the compound 3,11-difluoro-6,8,13-trimethyl-8H-quino[4,3,2-kl]acridinium methosulfate (RHPS4). In another embodiment the compound is BRACO-19. In another embodiment, the compound is an analog of BRACO-19 or RHPS4. In another embodiment, the cancer comprises cancer stem cells. In a further embodiment, the G-quadruplex ligand also induces proliferation of non-cancerous cells. In a further embodiment of the invention, the non-cancerous cell is a non-cancerous stem cell.

In a particular embodiment of the invention, the composition that is administered to an individual that has cancer comprising a cancer stem cell or that is suspected of having cancer comprising a cancer stem cell comprises at least one compound that causes reduction in telomere length in a cancer stem cell. In specific embodiments of the invention, the composition that is administered to an individual that has cancer comprising a cancer stem cell or that is suspected of having cancer comprising a cancer stem cell comprises a platinum compound. In a specific embodiment, the composition that is administered to an individual that has cancer comprising a cancer stem cell or that is suspected of having cancer comprising a cancer stem cell comprises cisplatin or Taxol.

In a particular embodiment of the invention, the composition that is administered to an individual that has cancer comprising a cancer stem cell or that is suspected of having cancer comprising a cancer stem cell comprises at least a first and a second compound that act synergistically to target telomeres in a cancer stem cell. In specific embodiments of the invention, said first compound is a G-quadruplex ligand. In a particular embodiment, said first compound is 3,11-difluoro-6,8,13-trimethyl-8H-quino[4,3,2-kl]acridinium methosulfate (RHPS4). In specific embodiments of the invention, said second compound is a platinum compound. In a specific embodiment, said second compound is cisplatin. In another specific embodiment, said second compound is carboplatin. In another embodiment the second compound is a mitotic spindle poison. In a specific embodiment the mitotic spindle poison is Paclitaxel, Vincristin, Vinblastion, Vinflunine, docetaxel, or epithiolones.

One embodiment of the invention includes a step comprising determining the presence of a cancer stem cell. A specific embodiment of the invention comprises detecting the presence of one or more specific cell surface markers that are present on cancer stem cells.

In particular embodiments the invention is drawn to compositions and methods of the invention for treating cancer comprising a cancer stem cell regardless of, for example, type or origin. Cancer comprising a cancer stem cell refers to, a pathophysiological state whereby a cell, including a stem cell, is characterized by dysregulated and/or proliferative cellular growth and the ability to induce said growth, either by direct growth into adjacent tissue through invasion or by growth at distal sites through metastasis in both, adults or children, and both, acute or chronic, including, but not limited to, carcinomas and sarcomas, such as, for example, acute lymphoblastic leukemia, acute myeloid leukemia, adrenocortical cancer, AIDS-related cancers, AIDS-related lymphoma, anal cancer, astrocytoma (including, for example, cerebellar and cerebral), basal cell carcinoma, bile duct cancer, bladder cancer, bone cancer, brain stem glioma, brain tumor (including, for example, ependymoma, medulloblastoma, supratentorial primitive neuroectodermal, visual pathway and hypothalamic glioma), cerebral astrocytoma/malignant glioma, breast cancer, bronchial adenomas/carcinoids, Burkitt's lymphoma, carcinoid tumor (including, for example, gastrointestinal), carcinoma of unknown primary site, central nervous system lymphoma, cervical cancer, chronic lymphocytic leukemia, chronic myelogenous leukemia, chronic myeloproliferative disorders, colon cancer, colorectal cancer, cutaneous T-Cell lymphoma, endometrial cancer, ependymoma, esophageal cancer, Ewing's Family of tumors, extrahepatic bile duct cancer, eye cancer (including, for example, intraocular melanoma, retinoblastoma, gallbladder cancer, gastric cancer, gastrointestinal carcinoid tumor, gastrointestinal stromal tumor (GIST), germ cell tumor (including, for example, extracranial, extragonadal, ovarian), gestational trophoblastic tumor, glioma, hairy cell leukemia, head and neck cancer, squamous cell head and neck cancer, hepatocellular cancer, Hodgkin's lymphoma, hypopharyngeal cancer, islet cell carcinoma (including, for example, endocrine pancreas), Kaposi's sarcoma, laryngeal cancer, leukemia, lip and oral cavity cancer, liver cancer, lung cancer (including, for example, non-small cell), lymphoma, macroglobulinemia, malignant fibrous histiocytoma of bone/osteosarcoma, medulloblastoma, melanoma, Merkel cell carcinoma, mesothelioma, metastatic squamous neck cancer with occult primary, mouth cancer, multiple endocrine neoplasia syndrome, multiple myeloma/plasma cell neoplasm, mycosis fungoides, myelodysplastic syndromes, myelodysplastic/myeloproliferative diseases, myeloma, nasal cavity and paranasal sinus cancer, nasopharyngeal cancer, neuroblastoma, non-Hodgkin's lymphoma, oral cancer, oral cavity cancer, osteosarcoma, oropharyngeal cancer, ovarian cancer (including, for example, ovarian epithelial cancer, germ cell tumor), ovarian low malignant potential tumor, pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, pregnancy and breast cancer, primary central nervous system lymphoma, prostate cancer, rectal cancer, retinoblastoma, rhabdomyosarcoma, salivary gland cancer, soft tissue sarcoma, uterine sarcoma, Sézary syndrome, skin cancer (including, for example, non-melanoma or melanoma), small intestine cancer, supratentorial primitive neuroectodermal tumors, T-Cell Lymphoma, testicular cancer, throat cancer, thymoma, thymoma and thymic carcinoma, thyroid cancer, transitional cell cancer of the renal pelvis and ureter, trophoblastic tumor (including, for example, gestational), unusual cancers of childhood and adulthood, urethral cancer, endometrial uterine cancer, uterine sarcoma, vaginal cancer, viral induced cancers (including, for example, HPV induced cancer), vulvar cancer, Waldenström's macroglobulinemia, Wilms' Tumor, and women's cancers. In a specific embodiment of the invention, the above mentioned cancers comprise cancer stem cells.

In particular embodiments the invention is drawn to compositions and methods of the invention for treating cancer regardless of cell type. In further embodiments, the cancer cell type is a cancer stem cell. A cancerous mass comprises a cancer stem cell and/or a plurality thereof, which is responsible for, inter alia, initiating and maintaining growth. Without being bound by theory, cancer stem cells may be more sensitive to telomere and/or telomerase targeted methods of treating cancer. Further, the invention is drawn to inducing the proliferation of non-cancer cells and/or non-cancer stem cells. One of skill in the art will realize the advantages to inhibiting cancer cell growth while encouraging non-cancer cell growth.

II. Combination Therapy

In certain embodiments of the invention, the G-quadruplex ligand is used in combination with other therapies. In a specific embodiment of the invention the combination therapy is another anti-cancer therapy.

In further embodiments, compositions of the invention are administered in combination with at least one compound that inhibits the transcription of reverse transcriptase subunit of the human telomerase gene (hTERT). Compounds that inhibit the transcription of reverse transcriptase subunit of the human telomerase gene (hTERT) that may be administered with the compositions of the invention include, but are not limited to, an arsenical compound, including, for example, sodium metaarsenite, GRN163L, and arsenic trioxide.

In further embodiments of the invention said G-quadruplex ligand, and/or said platinum compound may by administered separately, simultaneously or sequentially.

In one embodiment of the invention, the G-quadruplex ligand works additively or synergistically with another anti-cancer compound. In a specific embodiment of the invention, the additional anti-cancer compound is Paclitaxel, 17-AAG, heat shock protein inhibitors, gemcitabine or doxorubicin.

In yet further embodiments the invention is drawn to a pharmaceutical composition for the treatment of cancer in a subject in need of such treatment comprising a G-quadruplex ligand, or a platinum compound.

In further embodiments, compositions of the invention are administered in combination with a chemotherapeutic agent. Chemotherapeutic agents that may be administered with the compositions of the invention include, but are not limited to, antibiotic derivatives (e.g., doxorubicin (adriamycin), bleomycin, daunorubicin, and dactinomycin); antiestrogens (e.g., tamoxifen); antimetabolites (e.g., fluorouracil, 5-FU, methotrexate, floxuridine, interferon alpha-2b, glutamic acid, plicamycin, mercaptopurine, and 6-thioguanine); cytotoxic agents (e.g., carmustine, BCNU, lomustine, CCNU, cytosine arabinoside, cyclophosphamide, estramustine, hydroxyurea, procarbazine, mitomycin, busulfan, cis-platin, and vincristine sulfate); hormones (e.g., medroxyprogesterone, estramustine phosphate sodium, ethinyl estradiol, estradiol, megestrol acetate, methyltestosterone, diethylstilbestrol diphosphate, chlorotrianisene, and testolactone); nitrogen mustard derivatives (e.g., mephalen, chorambucil, mechlorethamine (nitrogen mustard) and thiotepa); steroids and combinations (e.g., bethamethasone sodium phosphate); and others (e.g., dicarbazine, asparaginase, mitotane, vincristine sulfate, vinblastine sulfate, etoposide, Topotecan, 5-Fluorouracil, paclitaxel (Taxol), Cisplatin, Cytarabine, and IFN-gamma, irinotecan (Camptosar, CPT-1), irinotecan analogs, and gemcitabine (GEMZAR™)).

In a specific embodiment, compositions of the invention are administered in combination with CHOP (cyclophosphamide, doxorubicin, vincristine, and prednisone) or any combination of the components of CHOP. In another embodiment, compositions of the invention are administered in combination with Rituximab. In a further embodiment, compositions of the invention are administered with Rituxmab and CHOP, or Rituxmab and any combination of the components of CHOP.

In one embodiment, the compositions of the invention are administered in combination with members of the tumor necrosis factor (TNF) family or antibodies specific for TNF receptor family members. TNF, TNF-related or TNF-like molecules that may be administered with the compositions of the invention include, but are not limited to, soluble forms of TNF-alpha, lymphotoxin-alpha (LT-alpha, also known as TNF-beta), LT-beta (found in complex heterotrimer LT-alpha2-beta), OPGL, FasL, CD27L, CD30L, CD40L, 4-1BBL, DcR3, OX40L, TNF-gamma (International Publication No. WO 96/14328), TRAIL, AIM-II (International Publication No. WO 97/34911), APRIL (J. Exp. Med. 188(6):1185 1190), endokine-alpha (International Publication No. WO 98/07880), TR6 (International Publication No. WO 98/30694), OPG, and neutrokine-alpha (International Publication No. WO 98/18921, OX40, and nerve growth factor (NGF), and soluble forms of Fas, CD30, CD27, CD40 and 4-IBB, TR2 (International Publication No. WO 96/34095), DR3 (International Publication No. WO 97/35904), TR5 (International Publication No. WO 98/30693), TR6 (International Publication No. WO 98/30694), TR7 (International Publication No. WO 98/41629), TRANK, TR9 (International Publication No. WO 98/56892), TR1O (International Publication No. WO 98/54202), 312C2 (International Publication No. WO 98/06842), and TR12, and soluble forms CD154, CD70, and CD153.

The compositions of the invention may be administered alone or in combination with other therapeutic or prophylactic regimens (e.g., radiation therapy, chemotherapy, hormonal therapy, immunotherapy, anti-tumor agents, anti-angiogenesis and anti-inflammatory agents). Such combinatorial therapy may be administered sequentially and/or concomitantly.

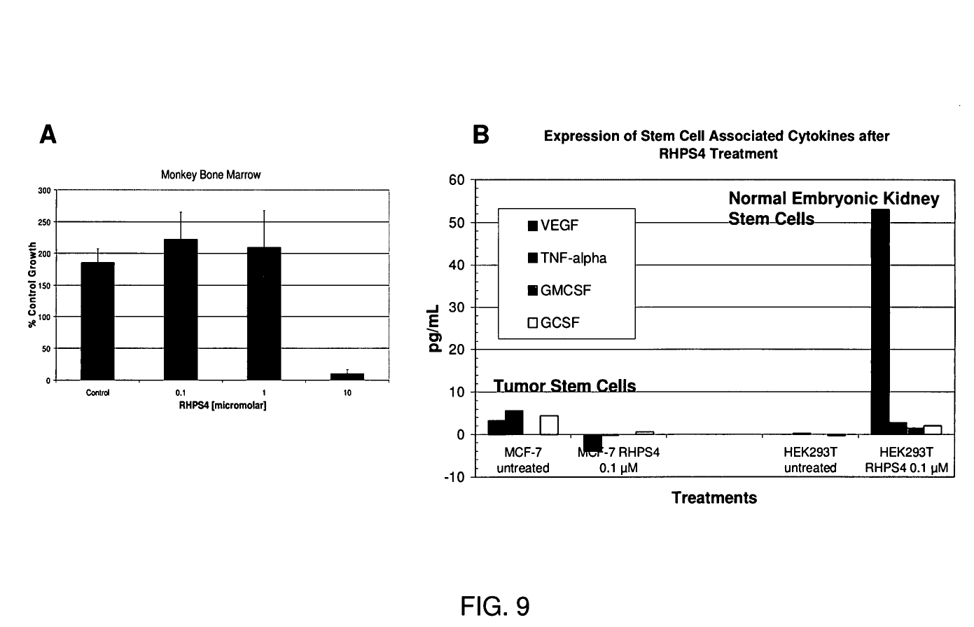

The invention also encompasses combining the compositions of the invention with other proposed or conventional hematopoietic therapies. Thus, for example, the compositions of the invention can be combined with compounds that singly exhibit erythropoietic stimulatory effects, such as erythropoietin, testosterone, progenitor cell stimulators, insulin-like growth factor, prostaglandins, serotonin, cyclic AMP, prolactin, and triiodothyzonine. Also encompassed are combinations of the invention with compounds generally used to treat aplastic anemia, such as, for example, methenolene, stanozolol, and nandrolone; to treat iron-deficiency anemia, such as, for example, iron preparations; to treat malignant anemia, such as, for example, vitamin B₁₂ and/or folic acid; and to treat hemolytic anemia, such as, for example, adrenocortical steroids, e.g., corticoids. See e.g., Resegotti et al., Panminerva Medica, 23:243 248 (1981); Kurtz, FEBS Letters, 14a:105 108 (1982); McGonigle et al., Kidney Int., 25:437 444 (1984); and Pavlovic-Kantera, Expt. Hematol., 8(supp. 8) 283 291 (1980), the contents of each of which are hereby incorporated by reference in their entireties. For example, as shown in the FIG. 9, RHPS4 could be used to stimulate hematopoiesis e.g. the expansion of normal hematopoietic stem cells or other normal stem cells. In an embodiment of the invention, the G-quadruplex ligand is used to stimulate hematopoiesis. In a specific embodiment of the invention, the G-quadruplex ligand is RHPS4.

Compounds that enhance the effects of or synergize with erythropoietin are also useful as adjuvants herein, and may be administered in combination with compositions of the invention. Such compounds include but are not limited to, adrenergic agonists, thyroid hormones, androgens, hepatic erythropoietic factors, erythrotropins, and erythrogenins. See e.g., Dunn, “Current Concepts in Erythropoiesis”, John Wiley and Sons (Chichester, England, 1983); Kalmani, Kidney Int., 22:383 391 (1982); Shahidi, New Eng. J. Med., 289:72 80 (1973); Urabe et al., J. Exp. Med., 149:1314 1325 (1979); Billat et al., Expt. Hematol., 10:135 140 (1982); Naughton et al., Acta Haemat, 69:171 179 (1983); Cognote et al. in abstract 364, Proceedings 7th Intl. Cong. of Endocrinology (Quebec City, Quebec, Jul. 17, 1984); and Rothman et al., 1982, J. Surg. Oncol., 20:105 108 (1982). Methods for stimulating hematopoiesis comprise administering a hematopoietically effective amount (i.e., an amount which effects the formation of blood cells) of a pharmaceutical composition containing compositions of the invention to a patient. The compositions of the invention may be administered to the patient by any suitable technique, including but not limited to, parenteral, sublingual, topical, intrapulmonary and intranasal, and those techniques further discussed herein. The pharmaceutical composition optionally contains one or more members of the group consisting of erythropoietin, testosterone, progenitor cell stimulators, insulin-like growth factor, prostaglandins, serotonin, cyclic AMP, prolactin, triiodothyzonine, methenolene, stanozolol, and nandrolone, iron preparations, vitamin B₁₂, folic acid and/or adrenocortical steroids.

In additional embodiments, the compositions of the invention are administered in combination with hematopoietic growth factors. Hematopoietic growth factors that may be administered with the compositions of the invention include, but are not limited to, LEUKINE™ (SARGRAMOSTIM™) and NEUPOGEN™ (FILGRASTIM™).

In further additional embodiments, the compositions of the invention are administered alone or in combination with an anti-angiogenic agent(s). Anti-angiogenic agents that may be administered with the compositions of the invention include, but are not limited to, Angiostatin (Entremed, Rockville, Md.), Troponin-1 (Boston Life Sciences, Boston, Mass.), anti-Invasive Factor, retinoic acid and derivatives thereof, paclitaxel (Taxol), Suramin, Tissue Inhibitor of Metalloproteinase-1, Tissue Inhibitor of Metalloproteinase-2, VEGI, Plasminogen Activator Inhibitor-1, Plasminogen Activator Inhibitor-2, and various forms of the lighter “d group” transition metals.

Lighter “d group” transition metals include, for example, vanadium, molybdenum, tungsten, titanium, niobium, and tantalum species. Such transition metal species may form transition metal complexes. Suitable complexes of the above-mentioned transition metal species include oxo transition metal complexes.

Representative examples of vanadium complexes include oxo vanadium complexes such as vanadate and vanadyl complexes. Suitable vanadate complexes include metavanadate and orthovanadate complexes such as, for example, ammonium metavanadate, sodium metavanadate, and sodium orthovanadate. Suitable vanadyl complexes include, for example, vanadyl acetylacetonate and vanadyl sulfate including vanadyl sulfate hydrates such as vanadyl sulfate mono- and trihydrates.

Representative examples of tungsten and molybdenum complexes also include oxo complexes. Suitable oxo tungsten complexes include tungstate and tungsten oxide complexes. Suitable tungstate complexes include ammonium tungstate, calcium tungstate, sodium tungstate dihydrate, and tungstic acid. Suitable tungsten oxides include tungsten (IV) oxide and tungsten (VI) oxide. Suitable oxo molybdenum complexes include molybdate, molybdenum oxide, and molybdenyl complexes. Suitable molybdate complexes include ammonium molybdate and its hydrates, sodium molybdate and its hydrates, and potassium molybdate and its hydrates. Suitable molybdenum oxides include molybdenum (VI) oxide, molybdenum (VI) oxide, and molybdic acid. Suitable molybdenyl complexes include, for example, molybdenyl acetylacetonate. Other suitable tungsten and molybdenum complexes include hydroxo derivatives derived from, for example, glycerol, tartaric acid, and sugars.

A wide variety of other anti-angiogenic factors may also be utilized within the context of the present invention. Representative examples include, but are not limited to, platelet factor 4; protamine sulphate; sulphated chitin derivatives (prepared from queen crab shells), (Murata et al., Cancer Res. 51:22 26, 1991); Sulphated Polysaccharide Peptidoglycan Complex (SP-PG) (the function of this compound may be enhanced by the presence of steroids such as estrogen, and tamoxifen citrate); Staurosporine; modulators of matrix metabolism, including for example, proline analogs, cishydroxyproline, d,L-3,4-dehydroproline, Thiaproline, alpha,alpha-dipyridyl, aminopropionitrile fumarate; 4-propyl-5-(4-pyridinyl)-2(3H)-oxazolone; Methotrexate; Mitoxantrone; Heparin; Interferons; 2 Macroglobulin-serum; ChIMP-3 (Pavloff et al., J. Bio. Chem. 267:17321 17326, 1992); Chymostatin (Tonkinson et al., Biochem J. 286:475 480, 1992); Cyclodextrin Tetradecasulfate; Eponemycin; Camptothecin; Fumagillin (Ingber et al., Nature 348:555 557, 1990); Gold Sodium Thiomalate (“GST”; Matsubara and Ziff, J. Clin. Invest. 79:1440 1446, 1987); anticollagenase-serum; alpha2-antiplasmin (Holmes et al., J. Biol. Chem. 262(4):1659 1664, 1987); Bisantrene (National Cancer Institute); Lobenzarit disodium (N-(2)-carboxyphenyl-4-chloroanthronilic acid disodium or “CCA”; (Takeuchi et al., Agents Actions 36:312 316, 1992); and metalloproteinase inhibitors such as BB94.

Additional anti-angiogenic factors that may also be utilized within the context of the present invention include Thalidomide, (Celgene, Warren, N.J.); Angiostatic steroid; AGM-1470 (H. Brem and J. Folkman J. Pediatr. Surg. 28:445 51 (1993)); an integrin alpha v beta 3 antagonist (C. Storgard et al., J. Clin. Invest. 103:47 54 (1999)); carboxynaminolmidazole; Carboxyamidotriazole (CAI) (National Cancer Institute, Bethesda, Md.); Conbretastatin A-4 (CA4P) (OXiGENE, Boston, Mass.); Squalamine (Magainin Pharmaceuticals, Plymouth Meeting, Pa.); TNP-470, (Tap Pharmaceuticals, Deerfield, Ill.); ZD-0101 AstraZeneca (London, UK); APRA (CT2584); Benefin, Byrostatin-1 (SC359555); CGP-41251 (PKC 412); CM101; Dexrazoxane (ICRF187); DMXAA; Endostatin; Flavopridiol; Genestein; GTE; ImmTher; Iressa (ZD1839); Octreotide (Somatostatin); Panretin; Penacillamine; Photopoint; PI-88; Prinomastat (AG-3540) Purlytin; Suradista (FCE26644); Tamoxifen (Nolvadex); Tazarotene; Tetrathiomolybdate; Xeloda (Capecitabine); and 5-Fluorouracil.

Anti-angiogenic agents that may be administered in combination with the compositions of the invention may work through a variety of mechanisms including, but not limited to, inhibiting proteolysis of the extracellular matrix, blocking the function of endothelial cell-extracellular matrix adhesion molecules, by antagonizing the function of angiogenesis inducers such as growth factors, and inhibiting integrin receptors expressed on proliferating endothelial cells. Examples of anti-angiogenic inhibitors that interfere with extracellular matrix proteolysis and which may be administered in combination with the compositions of the invention include, but are not limited to, AG-3540 (Agouron, La Jolla, Calif.), BAY-12-9566 (Bayer, West Haven, Conn.), BMS-275291 (Bristol Myers Squibb, Princeton, N.J.), CGS-27032A (Novartis, East Hanover, N.J.), Marimastat (British Biotech, Oxford, UK), and Metastat (Aeterna, St-Foy, Quebec). Examples of anti-angiogenic inhibitors that act by blocking the function of endothelial cell-extracellular matrix adhesion molecules and which may be administered in combination with compositions of the invention include, but are not limited to, EMD-121974 (Merck KcgaA Darmstadt, Germany) and Vitaxin (Ixsys, La Jolla, Calif./Medimmune, Gaithersburg, Md.). Examples of anti-angiogenic agents that act by directly antagonizing or inhibiting angiogenesis inducers and which may be administered in combination with compositions of the invention include, but are not limited to, Angiozyme (Ribozyme, Boulder, Colo.), Anti-VEGF antibody (Genentech, S. San Francisco, Calif.), PTK-787/ZK-225846 (Novartis, Basel, Switzerland), SU-101 (Sugen, S. San Francisco, Calif.), SU-5416 (Sugen/Pharmacia Upjohn, Bridgewater, N.J.), and SU-6668 (Sugen). Other anti-angiogenic agents act to indirectly inhibit angiogenesis. Examples of indirect inhibitors of angiogenesis which may be administered in combination with compositions of the invention include, but are not limited to, IM-862 (Cytran, Kirkland, Wash.), Interferon-alpha, IL-12 (Roche, Nutley, N.J.), and Pentosan polysulfate (Georgetown University, Washington, D.C.).

In particular embodiments, the use of compositions of the invention in combination with anti-angiogenic agents is contemplated for the treatment, prevention, and/or amelioration of cancers and other hyperproliferative disorders.

In further embodiments, the compositions of the invention are administered in combination with an antiviral agent. Antiviral agents that may be administered with the compositions of the invention include, but are not limited to, acyclovir, ribavirin, amantadine, and remantidine.

In certain embodiments, the compositions of the invention are administered in combination with antiretroviral agents, nucleoside/nucleotide reverse transcriptase inhibitors (NRTIs), non-nucleoside reverse transcriptase inhibitors (NNRTIs), and/or protease inhibitors (PIs). NRTIs that may be administered in combination with the compositions of the invention, include, but are not limited to, RETROVIR™ (zidovudine/AZT), VIDEX™ (didanosine/ddI), HIVID™ (zalcitabine/ddC), ZERIT™ (stavudine/d4T), EPIVIR™ (lamivudine/3TC), and COMBIVIR™ (zidovudine/lamivudine). NNRTIs that may be administered in combination with the compositions of the invention, include, but are not limited to, VIRAMUNE™ (nevirapine), RESCRIPTOR™ (delavirdine), and SUSTIVA™ (efavirenz). Protease inhibitors that may be administered in combination with the compositions of the invention, include, but are not limited to, CRIXIVAN™ (indinavir), NORVIR™ (ritonavir), INVIRASE™ (saquinavir), and VIRACEPT™ (nelfinavir).

In further embodiments, the compositions of the invention may be administered in combination with an antibiotic agent. Antibiotic agents that may be administered with the compositions of the invention include, but are not limited to, amoxicillin, aminoglycosides, beta-lactam (glycopeptide), beta-lactamases, Clindamycin, chloramphenicol, cephalosporins, ciprofloxacin, ciprofloxacin, erythromycin, fluoroquinolones, macrolides, metronidazole, penicillins, quinolones, rifampin, streptomycin, sulfonamide, tetracyclines, trimethoprim, trimethoprim-sulfamthoxazole, and vancomycin.

In other embodiments, the compositions of the invention may be administered in combination with anti-opportunistic infection agents. Anti-opportunistic agents that may be administered in combination with the compositions of the invention, include, but are not limited to, TRIMETHOPRIM-SULFAMETHOXAZOLE™, DAPSONE™, PENTAMIDINE™, ATOVAQUONE™, ISONIAZD™, RIFAMPIN™, PYRAZINAMIDE™, ETHAMBUTOL™, RIFABUTIN™, CLARITHROMYCIN™, AZITHROMYCIN™, GANCICLOVIR™, FOSCARNET™, CIDOFOVIR™, FLUCONAZOLE™, ITRACONAZOLE™, KETOCONAZOLE™, ACYCLOVIR™, FAMCICOLVIR™, PYRIMETHAMINE™, LEUCOVORIN™, NEUPOGEN™ (filgrastim/G-CSF), and LEUKINE™ (sargramostim/GM-CSF).

In additional embodiments, the compositions of the invention are administered alone or in combination with an anti-inflammatory agent. Anti-inflammatory agents that may be administered with the compositions of the invention include, but are not limited to, glucocorticoids and the nonsteroidal anti-inflammatories, aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, e-acetamidocaproic acid, S-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole, and tenidap.

The compositions of the invention may be administered alone or in combination with other adjuvants. Adjuvants that may be administered with the compositions of the invention include, but are not limited to, alum, alum plus deoxycholate (ImmunoAg), MTP-PE (Biocine Corp.), QS21 (Genentech, Inc.), BCG, and MPL. In a specific embodiment, compositions of the invention are administered in combination with alum. In another specific embodiment, compositions of the invention are administered in combination with QS-21. Further adjuvants that may be administered with the compositions of the invention include, but are not limited to, Monophosphoryl lipid immunomodulator, AdjuVax 100a, QS-21, QS-18, CRL1005, Aluminum salts, MF-59, and Virosomal adjuvant technology. Vaccines that may be administered with the compositions of the invention include, but are not limited to, vaccines directed toward protection against MMR (measles, mumps, rubella), polio, varicella, tetanus/diptheria, hepatitis A, hepatitis B, haemophilus influenzae B, whooping cough, pneumonia, influenza, Lyme's Disease, rotavirus, cholera, yellow fever, Japanese encephalitis, poliomyelitis, rabies, typhoid fever, and pertussis, and/or PNEUMOVAX-23™. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

In further embodiments, the compositions of the invention are administered in combination with an anticoagulant. Anticoagulants that may be administered with the compositions of the invention include, but are not limited to, heparin, warfarin, and aspirin. In a specific embodiment, the compositions of the invention are administered in combination with heparin and/or warfarin. In another specific embodiment, the compositions of the invention are administered in combination with warfarin. In another specific embodiment, the compositions of the invention are administered in combination with warfarin and aspirin. In another specific embodiment, the compositions of the invention are administered in combination with heparin. In another specific embodiment, the compositions of the invention are administered in combination with heparin and aspirin.

In further nonexclusive embodiments, the compositions of the invention are administered in combination with one, two, three, four, five, or more of the following drugs: NRD-101 (Hoechst Marion Roussel), diclofenac (Dimethaid), oxaprozin potassium (Monsanto), mecasermin (Chiron), T-714 (Toyama), pemetrexed disodium (Eli Lilly), atreleuton (Abbott), valdecoxib (Monsanto), eltenac (Byk Gulden), campath, AGM-1470 (Takeda), CDP-571 (Celltech Chiroscience), CM-101 (CarboMed), MI-3000 (Merckle), CB-2431 (KS Biomedix), CBF-BS2 (KS Biomedix), IL-1Ra gene therapy (Valentis), JTE-522 (Japan Tobacco), paclitaxel (Angiotech), DW-166HC (Dong Wha), darbufelone mesylate (Warner-Lambert), soluble TNF receptor 1 (synergen; Amgen), IPR-6001 (Institute for Pharmaceutical Research), trocade (Hoffman-La Roche), EF-5 (Scotia Pharmaceuticals), BIIL-284 (Boehringer Ingelheim), BIIF-1149 (Boehringer Ingelheim), LeukoVax (Inflammatics), MK-671 (Merck), ST-1482 (Sigma-Tau), and butixocort propionate (WarnerLambert).

In specific embodiments, the compositions of the invention are administered in combination with one, two, three, four, five or more of the following drugs: methotrexate, sulfasalazine, sodium aurothiomalate, auranofin, cyclosporine, penicillamine, azathioprine, an antimalarial drug (e.g., as described herein), cyclophosphamide, chlorambucil, gold, ENBREL™ (Etanercept), anti-TNF antibody, LJP 394 (La Jolla Pharmaceutical Company, San Diego, Calif.) and prednisolone.

In additional embodiments, the compositions of the invention may be administered in combination with cytokines. Cytokines that may be administered with the compositions of the invention include, but are not limited to, GM-CSF, G-CSF, IL2, IL3, IL4, IL5, IL6, IL7, IL10, IL12, IL13, IL15, anti-CD40, CD40L, IFN-alpha, IFN-beta, IFN-gamma, TNF-alpha, and TNF-beta. In further specific embodiments, compositions of the invention may be administered with any interleukin, including, but not limited to, IL-1 alpha, IL-1beta, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, and IL-22.

In further embodiments, the compositions of the invention are administered in combination with one or more chemokines. In specific embodiments, the compositions of the invention are administered in combination with an a (C—C) chemokine selected from the group consisting of gamma-interferon inducible protein-10 (gIP-10), interleukin-8 (IL-8), platelet factor-4 (PF4), neutrophil activating protein (NAP-2), GRO-a, GRO-b, GRO-g, neutrophil-activating peptide (ENA-78), granulocyte chemoattractant protein-2 (GCP-2), and stromal cell-derived factor-1 (SDF-1, or pre-B cell stimulatory factor (PBSF)); and/or a b(CC) chemokine selected from the group consisting of: RANTES (regulated on activation, normal T expressed and secreted), macrophage inflammatory protein-1alpha (MIP-1a), macrophage inflammatory protein-1beta (MIP-1b), monocyte chemotactic protein-1 (MCP-1), monocyte chemotactic protein-2 (MCP-2), monocyte chemotactic protein-3 (MCP-3), monocyte chemotactic protein-4 (MCP-4) macrophage inflammatory protein-1 gamma (MIP-1g), macrophage inflammatory protein-3 alpha (MIP-3a), macrophage inflammatory protein-3 beta (MIP-3b), macrophage inflammatory protein-4 (MIP-4/DC-CK-1/PARC), eotaxin, Exodus, and 1-309; and/or the g(C) chemokine, lymphotactin.

In specific further embodiment, the compositions of the invention are administered in combination with chemokine beta-8, chemokine beta-1, and/or macrophage inflammatory protein-4. In a specific embodiment, the compositions of the invention are administered with chemokine beta-8.

In order to increase the effectiveness of a G-quadruplex ligand, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver, et al., 1992). In the context of the present invention, it is contemplated that a G-quadruplex ligand could be used similarly in conjunction with chemotherapeutic, radiotherapeutic, or immunotherapeutic intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, gene therapy is “A” and the secondary agent, such as radio- or chemotherapy, is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

a. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, paclitaxel, gemcitabien, navelbine, farnesyl-protein transferase inhibitors, transplatinum, 5-fluorouracil, vincristin, vinblastin and methotrexate, or any analog or derivative variant of the foregoing.

b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

c. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with a G-quadruplex ligand. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

d. Genes

In yet another embodiment, the secondary treatment is a secondary gene therapy in which a second therapeutic polynucleotide is administered before, after, or at the same time as a G-quadruplex. Delivery of a vector encoding anyone of the following gene products may have a combined anti-hyperproliferative effect on target tissues. Alternatively, a single vector encoding two different genes may be used. A variety of proteins are encompassed within the invention, some of which are described below.

i. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

ii. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

iii. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

e. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

f. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adhesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

The compositions of the present invention and any functionally active derivatives thereof may be obtained by any suitable means. In specific embodiments, the derivatives of the invention are provided commercially, although in alternate embodiments the derivatives are synthesized. The chemical synthesis of the derivatives may employ well known techniques from readily available starting materials. Such synthetic transformations may include, but are not limited to protection, de-protection, oxidation, reduction, metal catalyzed C—C cross coupling, Heck coupling or Suzuki coupling steps (see for example, March's Advanced Organic Chemistry Reactions, Mechanisms, and Structures, 5^{th} Edition John Wiley and Sons by Michael B. Smith and Jerry March, incorporated here in full by reference.

III. Pharmaceutical Preparations

Pharmaceutical compositions of the present invention comprise an effective amount of one or more G-quadruplex ligand or additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one G-quadruplex ligand or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The G-quadruplex ligand may comprise different types of carriers depending on whether it is to be administered in solid, liquid or aerosol form, and whether it need to be sterile for such routes of administration as injection. The present invention can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The G-quadruplex ligand may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present invention, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art.

In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include G-quadruplex ligand, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the G-quadruplex ligand may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

A. Alimentary Compositions and Formulations

In certain embodiments of the present invention, the G-quadruplex ligand are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

B. Parenteral Compositions and Formulations

In further embodiments, G-quadruplex ligand may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

C. Miscellaneous Pharmaceutical Compositions and Formulations

In other certain embodiments of the invention, the active compound G-quadruplex ligand may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and laurocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroethylene support matrix is described in U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention. It is one of the objects of the present invention to provide methods, kits and compositions for treatment of cancer.

The following examples are offered by way of example, and are not intended to limit the scope of the invention in any manner.

### EXAMPLES

The following examples are included to demonstrate certain embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Exemplary Methods and Materials

RHPS4 was synthesized as described (Heald et al 2002). RHPS4 is water-soluble and was therefore dissolved in phosphate buffered saline (PBS). For in vitro studies paclitaxel (Taxol) was purchased from Sigma (St. Louis, Mo.) and dissolved in dimethylsulfoxide (DMSO); for in vivo experiments the clinical formulation was used and obtained Cremophor from Bristol-Myers Squibb, New York, N.Y.

The UXF1138L uterus carcinoma cell line was originally established from a patient tumor by Prof. Heiner Fiebig at the University of Freiburg, Germany (Fiebig and Burger, 2001). All animal experiments were conducted under an animal license approved by the German Federal Government (Regierungspräsidium Freiburg) and in compliance with the UKCCCR guidelines on experimental neoplasia (Workman et al., 1998). Six to eight weeks old female thymus aplastic nude mice of NMRI genetic background were used for establishment and serial propagation of the human tumor xenograft from the cell line. PC3 and MCF-7 cells were obtained from American Type Culture Collection, Manassas, Va. The HEK293T human embryonic kidney cell line was a kind gift from Dr. Arun Seth, Sunnybrook Health Sciences Centre, Toronto, CA. Umbellical vein cord blood was freshly obtained from a hospital maternity ward with the consent of the respective patient, specimens were anonymized. The cord blood was collected into a BD Vacutainer CPT™ and the mononuclear fraction isolated by centrifugation following the manufacturers instructions.

To prepare MTT proliferation assay and conduct in vitro combination studies, cells were grown under standard conditions (5% CO₂/37° C./humidified atmosphere) in their respective recommended media such as RMPI 1640, or DMEM (from Invitrogen, Carlsbad, Calif.) supplemented with 10% fetal calf serum and passaged routinely. Exponentially growing cells were seeded in 96-well plates (2,000/well) and drugs (RHPS4 or Paclitaxel) were added in concentrations ranging from 0.1 nM to 10 μM the following day. Cell proliferation was determined 5 days after continuous exposure to drug by addition of 3-(4,5-dimethylthiazol-2-yl)-2,5-diphenyltetrazoliumbromide (MTT) (Mosmann, 1983). The conversion of MTT to purple formazan by viable cells was measured using a SynergyHT plate reader (550 nm) and K4C software (BioTEK, Winooski, Vt.). Growth curves were generated as percent of control and growth inhibitory concentrations 50 and 100% determined.

Drugs were combined at the fixed ratio of their IC50 in six concentrations, ranging from 0.01 to 10 μM for RHPS4, and MTT assays were performed as described above. Fractions of affected cells were calculated from the absorbance readouts and entered into the Calcusyn 2.0 software (Biosoft, Ferguson, Mo.) (Chou and Talalay, 1984); combination index values were extracted.

To prepare metaphase spreads, cells were grown to 70% confluency and treated with 1 μM RHPS4 or PBS (vehicle control) for 24 hours in a T75 tissue culture flask. Supernatants were then replaced with media containing 10 μg/ml Colcemid (Sigma) and incubated for 90-120 minutes at 37° C. Next, cells were trypsinized and centrifuged at 500×g for 5 minutes; 8 ml 60 mmol/l KCl was added to the pellets and the cell suspension incubated for 20 minutes at 37° C. In a pre-fixation step, 2 ml freshly made fixative (methanol/glacial acid 3:1) was added on top of the hypotonic suspension and mixed carefully by turning the tube. After 10 minutes at RT, the mix was centrifuged at 600×g for 10 min. and the supernatant removed. For fixation, 10 ml fixative were added and the mix is kept at RT for 10 min. and centrifuged as above, the step was repeated 2 more times. Then, 0.5 ml fresh fixative was added to obtain a milky suspension of cells without clumps. Cleaned slides were placed horizontally at a 45° angle and 100 μl of cell solution dropped onto the slide from a distance of about 20 cm. Slides were dried at RT or directly dehydrated through an ethanol series of 70%, 90% and 100%. After dehydration, slides were rinsed in PBS and incubated with 0.1 μg/ml DAPI (4′, 6-diamidino-2-phenylindole)/PBS for 30 minutes at RT. To remove excess DAPI, slides are rinsed 4× with PBS and mounted (Vectashield, Burlingame, Calif.). Results were documented using the fluorescent module of a Leica DM4000 microscope with Retiga camera (Leica, Wetzlar, Germany).

To conduct telomere FISH (Fluorescence In Situ Hybridization), all human centromere (Cat. CP5095-B.5) and telomere (CP5097-DG.5) probes labelled with biotin or digoxigenin (Q-Biogene, Irvine, Calif.) were used for hybridization to metaphase preparations of UXF1138L cells following a protocol provided by the manufacturer. The probes were detected with fluorescein labelled avidin for centromere signal (green), and rhodamine labelled anti-digoxigenin for telomeres (red/pink). The chromosomes were counterstained with DAPI (blue). Images were captured at 100× magnification by using a Zeiss Axiovert Fluorescence Microscope (Carl Zeiss, Gottingen, Germany).

During phosphorylated H2AX (gamma-H2AX) and hTERT immunofluorescence staining, approximately 75,000 UXF1138L cells/chamber were seeded onto 8 chamber glass slides (Costar) 24 hrs prior to RHPS4 treatment. After exposure to 1 μM RHPS4 for 1, 6, or 24 hours, cells were washed twice with PBS and air dried. Cells were fixed and permeabilized by immersion into ice-cold methanol/acetone (1:1; 3×1 min). Slides were blocked overnight at 4° C. with 5% BSA in PBS and washed with PBS (3×) before incubation (2 hrs) with anti-gamma-H2AX mouse monoclonal antibody (Upstate, Waltham, Mass.; 1:250 in PBS) or hTERT monoclonal antibody (NCL-L-hTERT Novacastra, Newcastle, UK; 1:40) respectively. Control cells were probed with mouse IgG (Santa Cruz Biotechnolog Inc., Santa Cruz, Calif.). Slides were washed 3× with PBS, before incubation with a goat anti-mouse FITC conjugated secondary antibody (Sigma; 1:100, 3 h). Following further PBS washes (3×), slides were incubated with 1:5000 DAPI (2 mg/ml; Sigma), washed 3× in PBS and mounted with Vectashield mounting media. Images were visualized as described above.

During immunoblotting for gamma-H2AX, cells were grown to 50 to 80% confluent in 6-well plates (Falcon) and treated with 1 μM RHPS4 for 1, 6, and 24 hours. Histones were released by the method described by Meng et al. (2005). Briefly, cells were scraped and spun at 2-4° C./1000×g for 15 minutes. Pellets were washed twice with PBS, homogenized with 0.2N sulphuric acid and centrifuged for 15 minutes at 2-4° C./13,000×g. Supernatants were collected and 0.25 volume of 100% (w/v) trichloroacetic acid was added to precipitate histones. After centrifugation for 15 minutes at 2-4° C./13,000×g pellets were suspended in absolute ethanol for over night and again spun for 15 minutes at 2-4° C./13,000×g. Histones were dissolved in water and protein concentration was determined using the BioRad protein assay (BioRad Laboratories, Hercules Calif.). 12.5 μg of protein were loaded onto 4-20% Tris-glycine gels (Invitrogen) and separated at 125 volts for 90 min. Proteins were then transferred onto a polyvinylidene difluoride membrane (Immobilon-P, Millipore, Billerica, Mass.). Membranes were blocked with 10% non-fat milk in TBS-T (Tween²⁰ 0.02%) for 1 hour, followed by overnight incubation with gamma-H2AX (Upstate) antibody (1:1000 dil.). Signals were visualized by chemiluminescence using the ECL™ western blotting analysis system (Amersham Biosciences, Pittsburgh, Pa.). Coomassie blue staining was used to assure equal loading control.

For the first in vivo experiment, tumor fragments (5×5 mm) from untreated donor animals were implanted subcutaneously into both flanks of recipient mice. Treatment was initiated 6 days after transplantation (=day 0, median tumor volume of ˜70 mm³). Animals were randomized into groups following Lindner's randomization tables and treated by oral gavage with 5 mg/kg/d RHPS4 or vehicle (phosphate buffered saline) respectively (n=5-8 animals per group). In prior experiments, this dose was found to be the ½ maximal tolerated dose in the mouse strain used and was well tolerated in repetitive dosing regimens. Drug administration was repeated twice weekly for 8 times (Q3dx8) after randomization. Tumor growth was followed twice weekly by serial caliper measurement, body weights were recorded, and tumor volumes were calculated using the standard formula (length×width²)/2, where length is the largest dimension and width the smallest dimension perpendicular to the length (Alley et al., 2004; Geran et al., 1972). The median relative tumor volume was plotted against time. Relative tumor volumes were calculated for each single tumor by dividing the tumor volume on day X by that on day 0 (time of randomization). Growth curves were analyzed in terms of tumor inhibition (treated/control, T/C, calculated as median tumor weight of treated divided by median tumor weight of control animals times 100). Statistical data analysis was done using non-parametrical Wilcoxon Mann-Whitney statistics. Median relative tumor volumes of each treatment group were compared with those of the vehicle control groups. P values less than 0.05 were considered statistically significant. SPSS 2000, SYSTAT version 10 software was used.

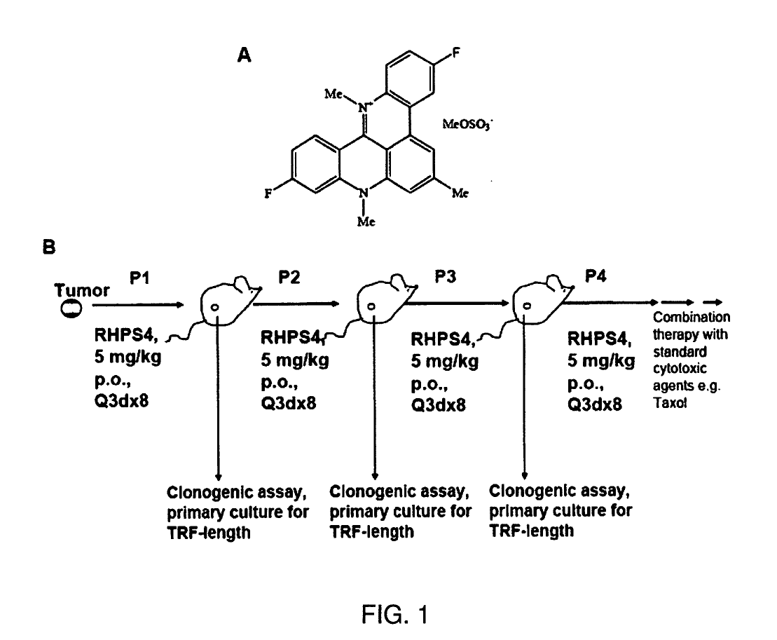

Upon termination of the experiment, which was when tumors reached a volume of 1.5 cm in diameter (day 28), RHPS4 treated tumor tissue and control tumors were excised, minced and digested using a mixture of collagenase (123 U/ml), DNase (375 U/ml), and hyaluronidase (290 U/ml) in RPMI 1640 medium at 37° C. for 3 hrs. All enzymes were purchased from Roche, Indianapolis Ind.). Primary cultures as well as clonogenic growth assays were prepared from the resulting single cell suspensions. Primary cultures were used for analysis of telomere length. In addition, RHPS4 treated tumors (5 mg/kg/d) and control were propagated into new animals for up to three times. The control mouse group was always derived from untreated tumor fragments, but from the same initial passage as were the RHPS4 treated tumors (FIG. 1B).

To conduct combination treatment with Paclitaxel, after 4 serial propagations of RHPS4 treated tumor tissue in nude mice (FIG. 1), RHPS4 was combined with Paclitaxel. Single agent Paclitaxel was given at 20 mg/kg i.v. on days 1 and 15. In combination with RHPS4 (given at 5 mg/kg p.o. twice weekly), only a single dose of Paclitaxel (20 mg/kg i.v.) was administered on day 1. Tumor growth parameters and body weight were assessed as described above.

Upon termination of the experiments, tumors from 3 mice per group were excised and immediately fixed in 10% PBS buffered formalin for 24 hours followed by routine paraffin embedding procedures (Burger et al. 2005).

Five-micrometer paraffin sections were cut, dewaxed, and antigen retrieval done in citrate buffer (pH 6.0) in the microwave for 30 minutes. Sections were then treated with methanol/3% hydrogen peroxide to remove endogenous peroxidase and blocked with 10% normal goat serum in PBS and stained. PBS was used as washing buffer. Cells were incubated overnight at 4° C. with a monoclonal anti-hTERT antibody (class IgG2a, kappa, Novacastra, Newcastle, UK) diluted 1:40 in PBS. Mouse immunoglobulin G2a isotype control (Santa Cruz) was used as negative control. hTERT-specific immunoperoxidase staining was developed using the DAKO Envision+ system (Envision 3,3V-diaminobenzidine Plus kit mouse, DAKO Cytomation). To enhance contrast, tissues were counterstained with hematoxylin. hTERT-specific staining intensity was documented using a Leica DM4000 microscope and digital camera. Sections were viewed and evaluated by two independent pathologists. Mean numbers of atypical mitoses were counted in control and treated tissues from 3 tumors (4 fields of 250 cells per tumor) for the 3 groups (Burger et al. 2005). Box plots were generated using SigmaPlot version 10 software and statistical significance between treatments calculated in SigmaPlot using the Student T-test.

In human tumor stem cell assay (HTCA)/clonogenic assay, single cell suspensions from in vivo studies above were washed in medium and passed through sieves and the resulting single cell suspensions seeded into soft agar (n=3 tumors per group) as described by us before (Fiebig et al., 2004). Single cell suspensions of cell lines were prepared by trypsinization from cells growing as monolayers on plastic. Briefly, 5,000 (HEK293T cells), 10,000 (PC3, MCF-7 and UXF1138L cells) or 50,000 (UXF1138L tumor tissue) vital cells were added to 0.2 ml Iscove's medium/20% fetal bovine serum/0.05% gentamycin containing 0.4% agar and plated on top of a base layer consisting of 0.2 ml medium with 0.75% agar. The next day, the agar layers were fed with Isocve's medium and cultures incubated at 37° C., 7% CO₂ for approximately 11 days. Vital colonies were stained with 2-(4-iodophenyl)-3-(4-nitrophenyl)-5-phenyltetrazolium chloride (1 mg/ml) 24 hrs before evaluation, and colonies >70 μm were counted with an automated image analysis system (Omincon FAS IV, BIOSYS GmbH, Karben, Germany). Drug effect was assessed as growth inhibitory concentrations 50 and 70% (IC₅₀, IC₇₀). Methylcellulose was used to grow cord blood stem cells instead of soft agar. The seeding density was 20,000 cells/well. Stem cell growth factor supplemented and optimized methylcellulose (Methocult H4434) was purchased from Stem Cell Technologies (Vancouver, CA). Methylcellulose lacking growth factors was used as negative growth control. Statistical significance between treatment groups was evaluated by using the Student T-test.

To measure TRF (Telomere Restriction Fragment)-length, genomic DNA was isolated from 3-7 day primary cultures established from single cell suspensions of control and treated UXF1138L xenograft tissues using the DNeasy Tissue Kit (Qiagen, Hilden, Germany). Southern blotting was performed with the Telo-TAGGG-telomere length kit from Roche (Penzberg, Germany) and analyzed as described before (Burger et al., 2005; Cookson et al., 2005).

### Example 2

### Effects of RHPS4 on Clonogenic Cell Growth In Vitro

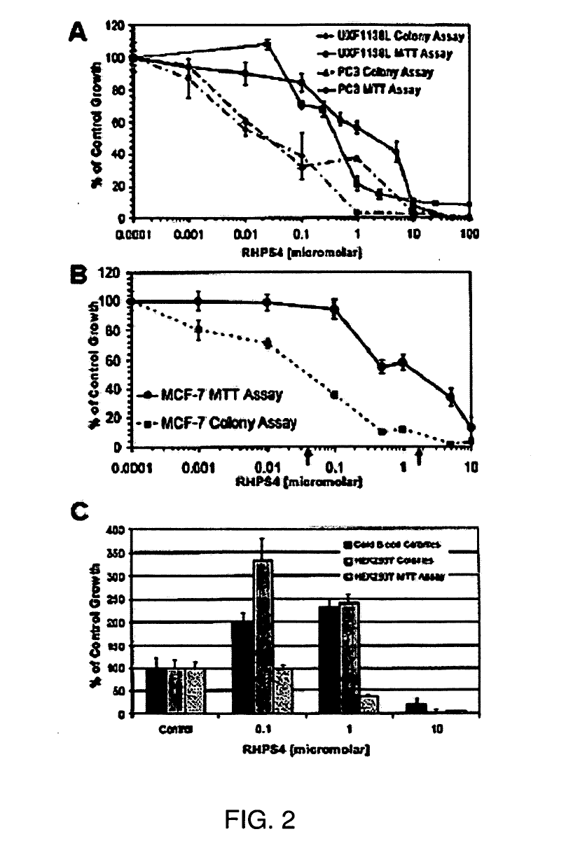

The growth inhibitory activity of RHPS4 in human bulk tumor cells have been compared, by MTT assay, against RHPS4 activity in tumor cells grown as colonies in the clonogenic assay, also termed the human tumor stem cell assay (HTCA) (FIG. 2A-B). HEK293T human embryonic kidney cells grown in the HTCA, and cord blood cells cultured in methylcellulose were also treated with RHPS4 (FIG. 2C). The MTT assay is a 5 day proliferation test measuring effects on a morphologically heterogeneous, differentiated cell population (bulk cells), whereas the HTCA and methylcellulose assays are longer term (10-15 day) tests in which only a very small fraction of a bulk culture (˜0.1-1%) will grow as colonies. Cells growing anchorage-independently as colonies in a semi-solid matrix are considered to be pluripotent stem cells (Hamburger & Salmon, 1977; Locke et al., 2005; Fiebig et al. 2004). FIG. 2A shows a comparison of responses to RHPS4 in two tumor cell lines with short telomeres, the uterus carcinoma UXF1138L, and the prostate cancer cell line PC3. Drug concentrations needed to inhibit colony growth in the HTCA were a magnitude of around 20 to 60-fold lower (IC50 UXF1138L=0.02 microM, PC3=0.03 mircoM) than those needed to cause 50% growth inhibition of the bulk population by MTT assay (IC50 UXF1138L=0.4 microM, PC3=1.8 microM). Similar observations were made with the breast cancer cell line MCF-7 (shown in FIG. 2B, HTCA IC50=0.04 microM; bulk cell IC50=2 microM). These data suggest that cancer stem cells are more sensitive to RHPS4 than the whole cancer cell population. Therefore, intriging evidence has been presented that RHPS4 can differentially inhibit the growth of clonogenic tumor cells, which are considered to be cancer stem cells.

### Example 3

### RHPS4's Effects on Normal Stem Cells

To assess RHPS4 effects on normal stem cells, the human embryonic kidney cell line HEK293T was exposed to drug in the MTT and HTCA assays, and tested RHPS4 effects on colony forming units of the mononuclear cell fraction of human cord blood in methyl cellulose (FIG. 2C). The cord blood colony assay was performed with and without colony stimulating growth factors, only methylcellulose containing growth factors grew colonies. Interestingly, RHPS4 concentrations that inhibited colony formation by human embryonic kidney and cord blood (>1 mircoM) cells were over 25-fold above those inhibiting tumor cell colony formation (FIG. 2C). Additionally, in normal cell types as compared to tumor cells, low and pharmacodynamically relevant concentrations of RHPS4 (0.01 to 1 microM) did induce colony formation (FIG. 2C). To assure that the induction of colony growth by RHPS4 in normal stem cells is reproducible, cord blood from three different individuals and HEK293T cells from different passages were used. Data shown in FIG. 2C represent the mean and standard deviation from three independent experiments. Consistently, 0.1 and 1 mircoM RHPS4 caused a stimulation of growth by doubling to tripling the number of colonies compared to vehicle (PBS) treated controls (FIG. 2C). However, the plating efficiency (actual number of colonies growing per total number of cells seeded) varied among the experiments and therefore the results are shown as % of control growth. E.g. HEK293T control colony growth ranged from a mean number of 54 to 463 colonies per well, but the least percentage (cut-off level) of growth induction by RHPS4 observed in either, the individual HEK293T or the cord blood experiments, was 150%. In contrast, HEK293T cells grown as monolayer cultures in the MTT assay showed no induction of growth at any of eight dose levels tested (0.001-50 mircoM). Instead, at RHPS4 levels that induced colony growth 2.4-fold (1 mircoM), bulk cell growth was inhibited to 60% of control (FIG. 2C).

As shown in FIG. 2C, colony forming units of cord blood and HEK293T cells were over a log-fold less sensitive to RHPS4 treatment than colonies forming from tumor cells (FIG. 2A-B). Cell kill of normal stem cells was only seen at high drug concentrations (˜10 mircoM) suggesting that RHPS4 might have a relatively wide therapeutic window. Moreover, at RHPS4 concentrations that markedly inhibited tumor colony forming units (0.1-1 mircoM) cord blood and HEK293T derived colony growth was induced. When clonogenic growth of human tumor cells was compared to bulk cell growth, pronounced differences were seen (FIG. 2A-B). Whole cell populations were more resistant to RHPS4. These observations strongly suggest that human tumor stem cells can be differentially targeted by G-quadruplex stabilizing ligands and are in agreement with recent findings that hTERT is a “stemness” gene: hTERT over-expression has been found to promote stem cell mobilization, whereas short telomeres have been reported to cause stem cell failure (Sarin et al., 2005; Hao et al., 2005). Therefore, the loss of telomere-associated proteins that have capping function such as hTERT and the stabilization of G-quadruplexes at the telomeric G-strand overhang upon ligand binding is more detrimental to cancer cells than normal cells that express telomerase.

### Example 4

### Effects of RHPS4 on Bulk Tumor and Clonogenic Tumor Cell Growth In Vivo

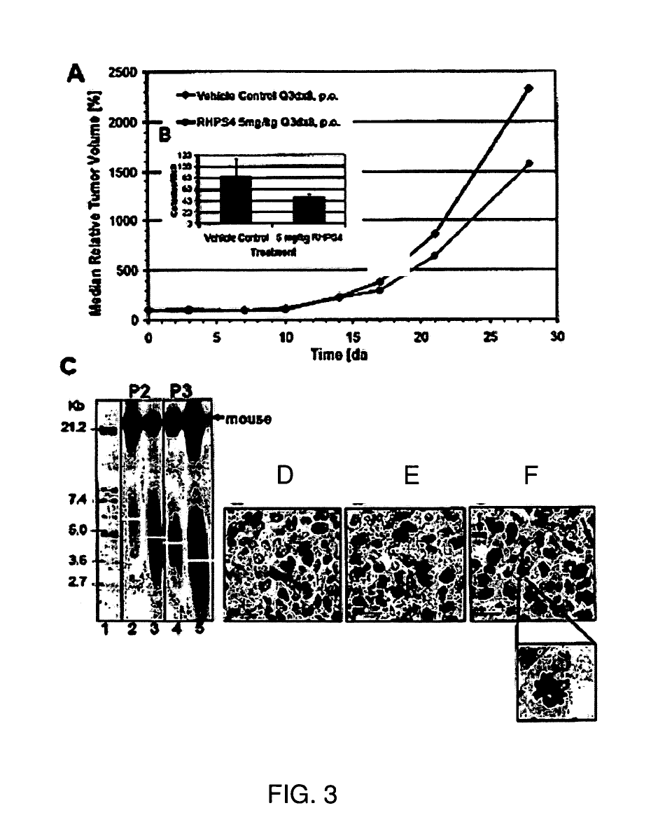

RHPS4 was administered orally twice a week for the course of the experiment at half of its maximal tolerated dose (5 mg/kg/d). 5 mg/kg/d RHPS4 was well tolerated in all in vivo studies and did not cause any noticeable side effects such as body weight loss (Table 2). Efficacy of RHPS4 in subcutaneously growing UXF1138L xenografts was determined in terms of “bulk” tumor growth inhibition relative to vehicle treated controls (FIG. 3A) as well as by measuring clonogenicity. As shown in FIG. 1, control treated and RHPS4 treated UXF1138L tumors were transplanted into new animals upon termination of a therapy experiment and treatment was essentially continued in another host. Engraftments of treated tumor tissues were performed for 4 consecutive passages. The result for single agent RHPS4 in passages 1-4 are summarised in table 2. Because UXF1138 xenografts are fast growing (average tumor doubling time=5 days), it is necessary to employ serial transplantation of RHPS4 treated tissues in order to evaluate pharmacodynamic endpoints that would likely require “chronic” drug exposure such as successive telomere erosion and inhibition of G₀-arrested tumor stem cell fractions. The results of the single agent study in passage 3, are shown in FIG. 3A. Although, RHPS4 did not show oral single agent activity in any of the 4 passages, it was observed that there were marked reduction in clonogenicity of RHPS4 treated tumor tissue in the soft agar tumor stem cell assay; inhibition of stem cell growth increased with successive passages (Table 2). In the experiment depicted in FIG. 3, it was found that tumor growth inhibition amounts to a maximal extent of 33% (optimal T/C at day 28 was 67%, p=0.02) compared to control, but a significant inhibition of colony forming units in the same tumor tissues of 54%±6.6 (p<0.0028) (FIG. 3A, insert).

TABLE 2
Summary of in vivo efficacy and pharmacodynamics

| | opt. Test/ | | | | | | |
|---|---|---|---|---|---|---|---|
| UXF1138L | Control | | BWC | Deaths | HTCA | | TRF |
| Xenograft | [%] | (day) | [%] | [n/n] | growth [%] | | [kb] |
| Passage #1 | | | | | | | |
| Vehicle Control | 100 | (0) | +9 | 0/5 | 100 ± 5.2 | | 6.0 |
| RHPS4 5 mg/kg | 63 | (16) | +26 | 0/5 | 83 ± 2.3 | | 5.1 |
| Passage #2 | | | | | | | |
| Vehicle Control | 100 | (0) | +13 | 0/6 | 100 ± 22 | | 5.7 |
| RHPS4 5 mg/kg | 100 | (7) | +9 | 0/6 | 93 ± 2.3 | | 4.7 |
| Passage #3 | | | | | | | |
| Vehicle Control | 100 | (0) | +29 | 0/8 | 100 ± 35 | | 4.6 |
| RHPS4 5 mg/kg | 67.7 | (28) | +21 | 0/8 | 54.5 ± 6.6 | | 3.4 |
| Passage #4 | | | | | | | |
| Vehicle Control | 100 | (0) | +22 | 0/5 | 100 ± 6.2 | | 4.9 |
| RHPS4 5 mg/kg | 62 | (16) | +17 | 0/5 | 44 ± 6.4 | | 4.2 |
| | | | | | CR | PR | P |
| Paclitaxel 20 mg/kg | 8 | (24) | +6 | 0/6 | 2/12 | 7/12 | 3/12 |
| Paclitaxel/RHPS4 | 0 | (19) | +10 | 0/5 | 8/10 | 2/10 | 0/10 |

Opt. Test/Control [%] (d), optimal test/control median tumor volume in % and day it was observed; BWC, maximal median body weight change in %; [n/n], number of drug related death per number of mice per group; HTCA, growth in the human tumor colony assay, colony growth of control was set 100%; TRF, mean telomere restriction fragment length in kilo bases; CR, complete remission; PR, partial regression at any time during the experiment compared to initial tumor volume; P, progression.

### Example 5

### Single Agent RHPS4 Modulates Telomeres and Telomerase In Vivo

DNA generated from primary cultures of RHPS4 treated tumor tissues that were harvested at termination of each experiment (see FIG. 1), was analyzed for telomere length (Table 2). As shown for passages 2 and 3 a clear difference between TRF-length of control and treatment groups was observed (FIG. 3C). The mean telomere length in RHPS4 treated xenograft tissue was approximately 1 kb lower than in control tissues (Table 2). Overall, TRF-length appeared to shorten at a rate of 1 kb per passage (˜28 days). It has to be noted that accurate measurement of telomere length of primary cultures from xenografts is problematic, because the cultures contain a mix of human cancer cells and murine cells. As seen in FIG. 3C, an additional strong very high TRF signal (>21 Kb) representing mouse telomeres was detected. Compared to the TRF length of pure human UXF 1138L cells growing in tissue culture (2.7 Kb), the primary cells derived from in vivo grown UXF1138L tumors, had longer telomeres that varied in control cultures from passage to passage (Table 2, FIG. 3C). This is likely due to contamination with mouse cells.

### Example 6

### hTERT Protein Expression in Control and Treated UXF1138L Xenograft Tissues

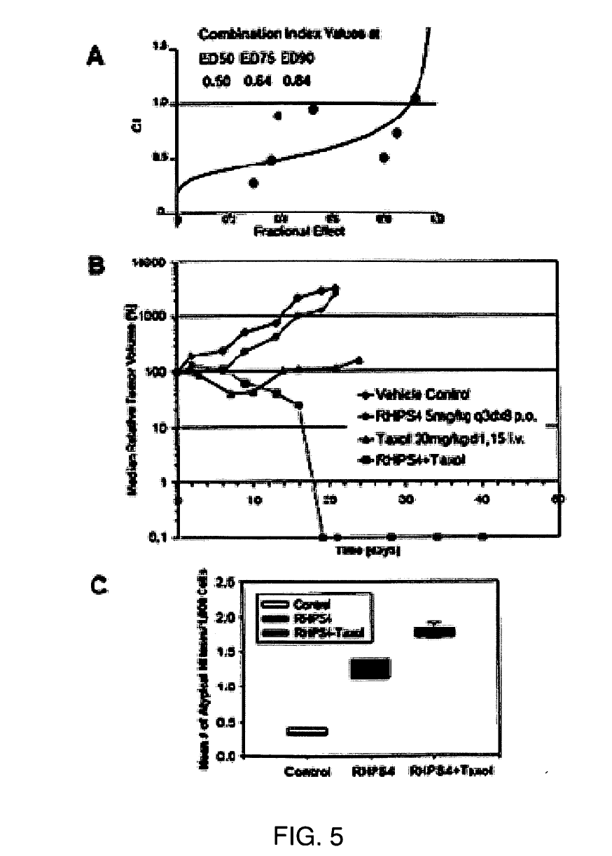

Control and treated UXF1138L xenograft tissues were also analyzed for hTERT protein expression (FIG. 3D-F). Control tumor tissue (FIG. 3E) readily expressed nuclear hTERT with an accumulation of the enzyme in the nucleoli. In RHPS4 treated UXF1138L xenograft tissue, loss of strong nuclear hTERT expression was observed, but weak nuclear and cytoplasmic hTERT staining remained (FIG. 3F). Isotype control antibody stained sections were completely negative (FIG. 3D), confirming that the weak hTERT protein expression is specific. Reduced hTERT expression was accompanied by the prominent occurrence of atypical mitotic figures such as ring chromosomes (FIG. 3F, enlargement) and anaphase bridges, indicative of telomere dysfunction and chromosomal damage. Atypical mitotic figures were quantified in FIG. 5C. RHPS4 mono therapy (5 mg/kg/d p.o.) evoked a significant induction of mitotic abnormalities compared to vehicle treated control (p=0.0011),

### Example 7

### Telomere Uncapping by RHPS4 In Vitro

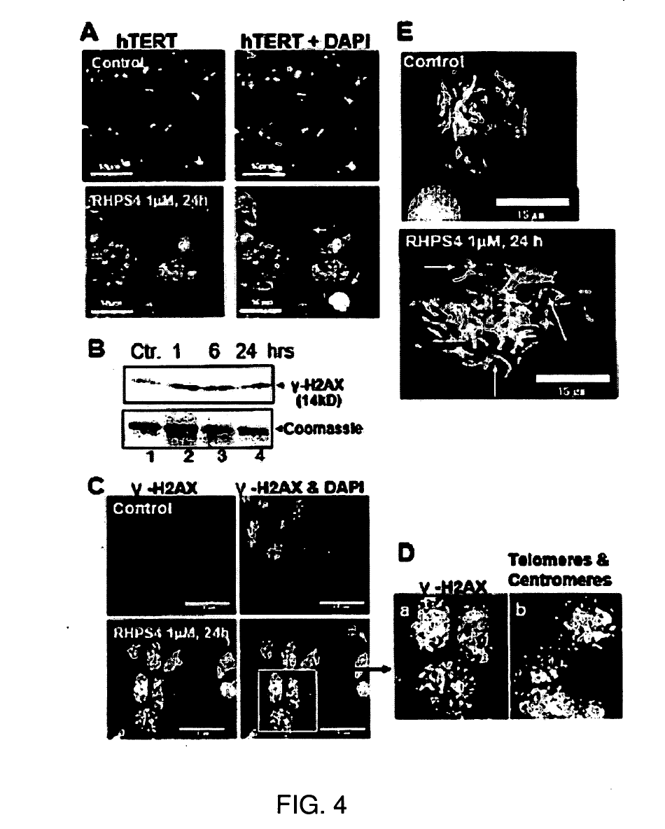

To confirm and clarify the data presented in FIG. 3D, hTERT protein expression was followed after treatment with 1 μM RHPS4 in UXF1138L cells in vitro. Control cells exhibited strong expression of hTERT in the nucleoplasm particularly in the nucleoli (FIG. 4A); nuclear hTERT expression was attenuated, whereas cytoplasmic protein was more detectable in cells treated with RHPS4 for 24 hrs (FIG. 4A, white arrows). This suggests that RHPS4 binding to the telomere can displace hTERT from the nucleus leading to its translocation into the cytoplasm. Concomitantly, the phosphorylation of histone variant H2AX, gamma-H2AX (FIG. 4B-C) was observed, which indicates putative telomere-initiated DNA-damage signalling. The data showing the loss of the telomerase catalytic subunit hTERT from the nucleus (FIG. 4A) and the rapid induction of putative telomere-initiated DNA-damage signalling as indicated by gamma-H2AX phosphorylation. gamma-H2AX expression was seen as early as 1 hour after exposure of UXF1138L cells to 1 μM RHPS4 by Western blot and at similar levels at 6 and 24 hours (FIG. 4B), suggesting the maximal signal was reached at 1 hr already. The 24 hour time point by immunofluorescence staining of gamma-H2AX foci (FIG. 4C) was confirmed. The majority of RHPS4 treated UXF1138L cells showed strong gamma-H2AX foci formation that were extended throughout the nucleus (FIG. 4D, left panel), a smaller fraction of nuclei showed a distinct punctuate gamma-H2AX pattern (FIGS. 4C and D). To investigate whether gamma-H2AX foci might localize to telomeres, fluorescence in situ hybridizations with telomere and centromere probes on interphase nuclei of UXF1138L cells (FIG. 4D, right panel) were performed. Because of the very short telomere length in UXF1138L cells, telomere signal (pink, FIG. 4D) was very weak, but a clear punctuate pattern was observed that did not match to the more diffuse and extensive gamma-H2AX foci. Moreover, the number of centromere (green) and telomere signals (pink, FIG. 4D) was consistent with the number of chromosomes, whereas gamma-H2AX foci exceeded the number of telomeres.

During the microscopic evaluation of gamma-H2AX foci, it became apparent that RHPS4 treated UXF1138L had an increased occurrence of anaphase bridges (data not shown). To test whether anaphase bridges are a result of chromosome fusions, metaphase spreads were generated from control cells and cells treated with 1 μM RHPS4 for 24 hours (FIG. 4E). DAPI staining revealed that RHPS4 has a marked effect on chromosome morphology; an increase in end-to-end joining, as evident in ring and dicentric chromosomes were observed (FIG. 4E, white arrows). The very rapid occurrence of RHPS4 effects depicted in FIG. 4, strongly support the hypothesis that RHPS4 can cause telomere capping alterations in tumor cells with short telomeres such as UXF1138L. Exposure to 1 microM RHPS4 for 24 hrs led to a marked increase in end-to-end joining, as evident in ring and dicentric chromosomes in metaphase spreads compared to vehicle controls. This earlier response to telomere dysfunction by UXF1138L cells compared to melanoma and prostate cancer cell lines might be due to their very short telomeres (2.7 Kb).

### Example 8

### Synergistic Effects of RHPS4 and Paclitaxel

The uterine carcinoma UXF1138L xenograft used in this study is overall resistant to standard chemotherapy including drugs that are substrates of Pgp and BCRP such as doxorubicin and mitoxantrone (Fiebig and Burger, 2001); only Paclitaxel has single agent activity and tumors inevitably re-grow after treatment (FIG. 5B). This indicates that UXF1138L tumors contain cells that can escape cytotoxic therapy and re-populate the tumor consistent with the existence of cancer stem cells. Under the influence of a mitotic spindle poison (Paclitaxel stabilizes microtubles), mitotic cells fail to enter anaphase. This mechanism together with the telomeric DNA-damage response induced by RHPS4, which leads to anaphase bridging (see FIG. 4E), suggested that the two agents might synergize. First, in vitro cytotoxicity assays were performed in UXF1138L cells with the single agents and the combinations thereof at their fixed IC50s, and processed the results using Calcusyn software. Paclitaxel combined with RHPS4 showed combination indices (CI) below 1 at all levels (%) of effect, ED50 (ED, effective dose), ED75 and ED90, indicating synergism of the two drugs (FIG. 5A). Second, RHPS4 was combined with Paclitaxel in vivo and evaluated UXF1138L tumor growth inhibition in nude mice. The study was performed with UXF1138L tumors in passage 4 (FIG. 1B) of continuous treatment with RHPS4 and not previously untreated UXF1138L tumors because the wish was to continue to study single agent activity with successive passages and exploit the concept of RHPS4 as a chemosensitizing agent; RHPS4 was given as detailed above (see FIG. 3). The combination of RHPS4 and Paclitaxel together showed markedly enhanced efficacy over that of either single agent alone (FIG. 5B, Table 2). Paclitaxel alone produced significant growth inhibition (optimal T/C [day 21]=8%, p<0.04) with transient remissions seen on days 7-10 when the drug was given i.v. on days 1 and 15. RHPS4 single agent activity was slightly more pronounced than in passage 3 (FIG. 3) with an optimal T/C of 62% (FIG. 5B). For the in vivo combination, RHPS4 was administered at 5 mg/kg p.o. twice weekly till the experiment was terminated (day 40, FIG. 5B) and injected Paclitaxel i.v. (20 mg/kg=MTD) together with the first dose of RHPS4. A second dose of Paclitaxel on day 15 was not given, because the tumors had regressed (T/C day 15=1%, FIG. 5B). Complete remissions were observed as of day 19 (T/C=0%, p<0.0017). The combination regimen and both of the single agents were well tolerated and appeared to lack noticeable, side effects. No body weight loss or drug-related deaths were observed (Table 2). Groups of 5-6 animals with two subcutaneously growing xenografts each (n=10-12 tumors) were used. Individual animals in the combination group had residual tumor masses (smaller than the tumor size at day 0 of the experiment, Table 2), which were excised and analyzed for mitotic abnormalities. UXF1138L vehicle control tumors and xenografts treated with RHPS4 alone were also examined. As seen before for the single agent treatment, anaphase-bridging and atypical mitoses occurred (FIG. 3D, 5C, p<0.001). They were even more pronounced in the combination group (FIG. 5C, p<0.0003). Taken together, the in vitro and in vivo data suggest that Paclitaxel and RHPS4 could be useful clinical combination partners. Effective tumor debulking by Paclitaxel together with eradication of cancer stem cells by RHPS4 could explain the marked synergism of these two agents against UXF138LX xenografts in vivo.

### Example 9

Chemotherapy and/or radiation can miss cancer stem cells leading to the repopulation of therapy resistant cancer cells. However, targeting drug efflux pumps and surface markers directly eliminates cancer stem cells that self-renew and induce differentiation and thereby reduces the chances of repopulation of cancer cells. In one embodiment of the invention, the elimination of cancer stem cells reduces the risk of the cancer reoccurring. FIG. 6 demonstrates the need for targeting cancer stem cells.

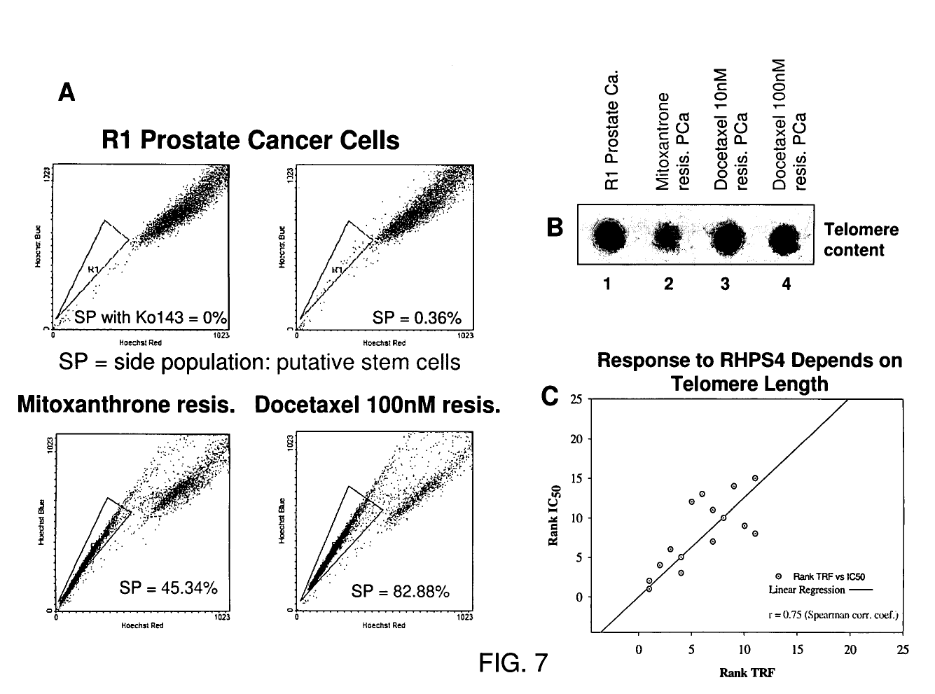

In one embodiment of the invention, cancer stem cells have shorter telomeres than other cancer cells (FIG. 7). Side population analysis was done to distinguish between cancer stem cells in R1 prostate cancer cells and non-stem cancer stem cells. A primitive CD34^{low/neg} stem cell population has been defined in normal bone marrow, which has the unique capacity to efflux lipophilic dyes such as Hoechst 33342 as a result of high levels of expression of P-glycoprotein (MDRa/ABCB1) transporter and BCRP (breast cancer resistance protein, ABCG2). This cell population has been shown to function as stem cells in bone marrow and has been termed the “side population” (SP); it is also found in cancer cell populations where it has tumor initiating properties (Hirschmann-Jax et al., 2004; Zhou et al., 2001). R1 is the parental prostate cancer cell line with a SP of 0.36% of cells of the total population (100%). The identity of the SP has been affirmed by using the BCRP efflux pump specific blocker Ko143, which abolishes the SP in R1 cells. Subclones of the R1 chemosensitive prostate cancer cell line that are resistant to the clinically used cytotoxic agents mitoxantrone and docetaxel, show a much expanded SP of 45% and 83% of cells respectively. In fact docetaxel resistant R1 cells are almost all stem cell like cells. This suggests that the induction of resistance to currently available chemotherapeutic drugs for the treatment of prostate cancer leads to an increase of difficult to treat cancer stem cells.

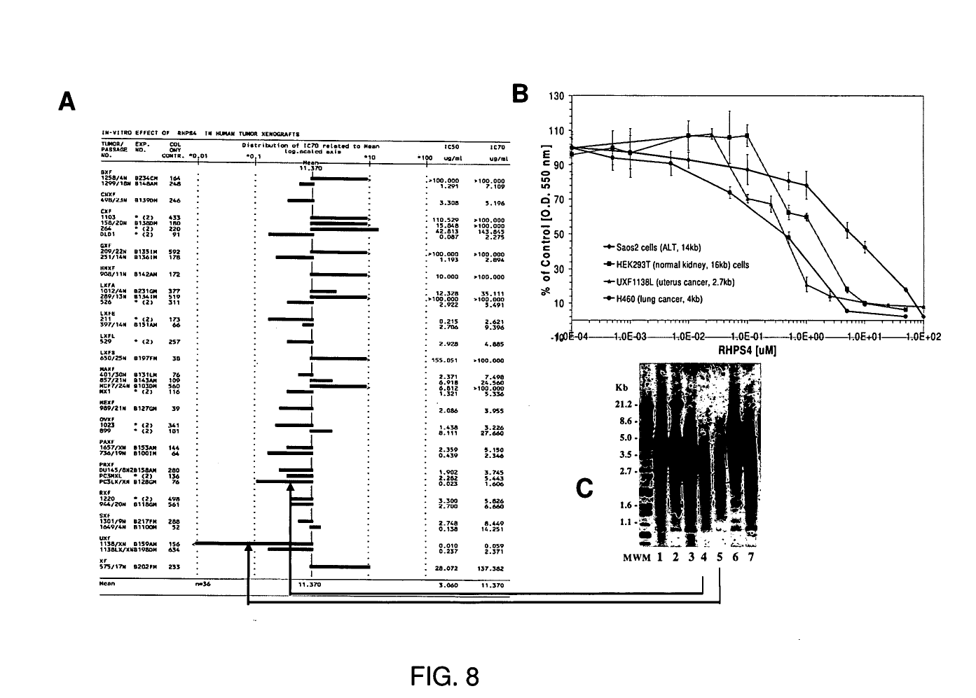

Low telomere content correlates with a short telomere length. Genomic DNA was isolated from prostate cancer cell lines by using a DNA extraction kit (Quiagen). DNA was precipitated overnight at −80° C. from the aqueous solution and the precipitate was dissolved in 10 μl water, denatured at 95° C. for 10 min, and 250 ng DNA was spotted onto Hybond membranes (GE Healthcare, Piscataway, N.J.) with a Schleicher & Schuell apparatus (Whatman, Sanford, Me.). Membranes were then developed using the TeloTAGG telomere length assay kit (Roche). It was found that the mitoxantrone and docetaxel resistant subclones of R1 had substantially lower telomere content than the parental cells indicating that they have shorter telomeres and that this might make these cells susceptible to telomere targeting agents such as RHPS4 (see FIG. 7C). The data further suggest that the short telomere length might result from the large fraction of stem cell like cells (SP) in these subclones. The data show that the shorter the telomeres of a cancer cell the more sensitive are these cells to the telomere targeting agent RHPS4 (FIG. 7C). Overall, the more sensitive tumor types in the stem cell assay toward RHPS4 treatment has shorter telomeres, the more resistant tumors have longer telomeres (FIG. 8B).

In an embodiment of the invention, tumors with shorter telomeres are more sensitive to RHPS4 (FIG. 8). Growth data were generated using the human tumor stem cell/clonogenic assay. The clonogenic assay was performed with xenograft tissues only. Xenografts growing s.c. in nude mice were removed when an average diameter of 1.5 cm was reached, they were mechanically disaggregated and subsequently incubated with collagenase (123 U/ml), DNase (375 U/ml) and hyaluronidase (290 U/ml) in RPMI 1640 at 37° C. for 30 minutes. Cells were washed and passed through sieves. The clonogenic assay was performed in 24-well plates according to a modified two-layer soft agar assay (Hamburger and Salmon, 1977). Cells were added in 0.2 ml ISCOVES medium/20% FBS containing 0.4% agar and plated on top of the base layer (0.75% agar). After 24 h drug was added (0.01-100 μM) in additional 0.2 ml of medium. Cultures were incubated at 37° C., 7% CO₂ for 15 days and monitored closely for colony growth. Vital colonies were stained with 2-(4-iodophenyl)-3-(4-nitrophenyl)-5-phenyltetrazolium chloride (1 mg/ml) 24 hours prior to evaluation and colonies >50 μm were counted with an automated image analysis system (OMNICON FAS IV, Biosys GmbH). Drug effects were assessed in terms of growth inhibitory concentrations 50 and 70% (IC₅₀ and IC₇₀ values). There is a two log-fold difference in the inhibitory concentrations 50% of RHPS4 in cell lines with short telomeres compared to those with long telomeres (FIG. 9B). Saos-2 cells are osteosarcoma cells that do not have telomerase, but instead maintain their very long telomeres through an alternative lengthening mechanism (ALT). HEK293T cells are normal embryonic kidney cells.

Telomere length was determined by Southern blotting. Mean telomere restriction fragment length (TRF-length) was determined using the Telo-TAGGG-telomere length kit from Roche (Penzberg, Germany), following the manufacturer's instructions. Genomic DNA was isolated from pellets of permanent cell lines and primary cells grown in culture with 0.5 and 1 μM RHPS4 for 15 days and cells grown in vehicle treated medium (PBS). DNA (2 μg) digested with HinfI and RsaI (2 h 37° C.), was separated on a 0.8% agarose gel in 1×TAE buffer. Telomere length for tumors available as xenograft material only, was measured using DNA derived from primary cultures.

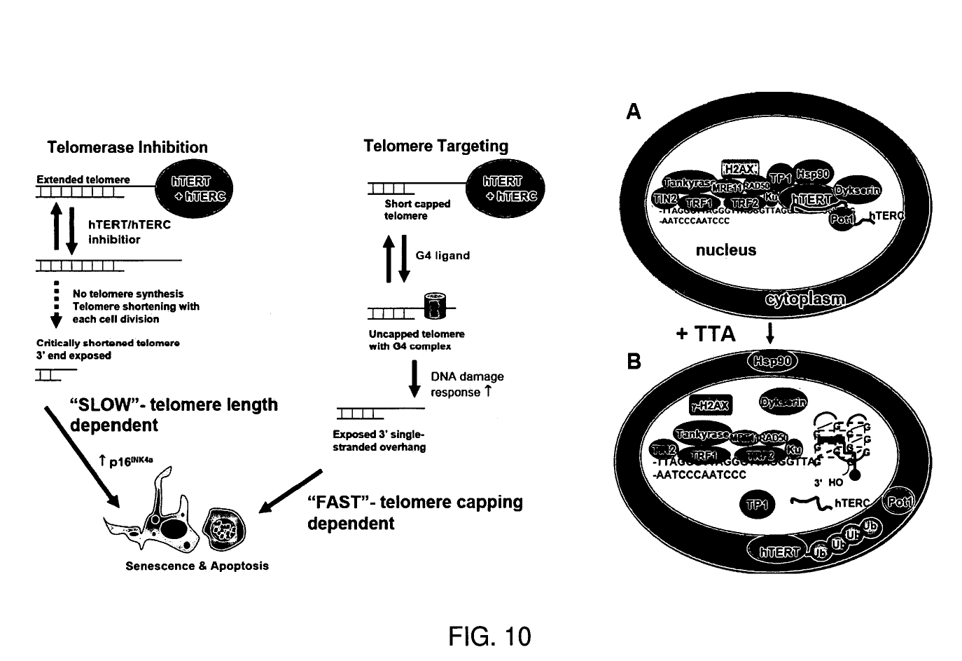

In a further embodiment of the invention, telomerase inhibition with a hTERT/hTERC inhibitor leads to slow telomere length dependent senescence and apoptosis. In another embodiment of the invention telomere targeting with G4 ligand leads to senescence and apoptosis. In another embodiment both telomerase inhibition and telomere targeting lead to further senescence and apoptosis of cancer cells (FIG. 10).

In one embodiment of the invention, RHPS4 induces the growth of normal stem cells at low concentrations and this is associated with an induction of cytokines (FIG. 9). Analysis of RHPS4 with normal monkey bone marrow stem cells demonstrated that RHPS4 induced the growth of normal monkey bone marrow stem cells at low drug concentrations that killed cancer stem cells (FIG. 9A). For the monkey bone marrow and the human cord blood, methylcellulose from StemCell Technologies (Vancouver) was used to grow the stem cells as colonies. An expression analyses of stem cell associated cytokines after RHPS4 treatment also demonstrated that RHPS4 inhibits the secretion of stem cells associated cytokines by breast tumor stem cells, but induced cytokine secretion in normal stem cells (FIG. 9B). Cancer tissues are composed of various cell types, tumor cells, stromal fibroblasts, blood vessels, inflammatory cells, and adipocytes, all of which can carry over into single cell suspensions that are seeded into soft agar after tissue digestion. To examine whether tumor stem cells secrete cytokines that support their growth of enforce differentiation, the expression of 6 cytokines found in the supernatant of clonogenic assays was evaluated. Ultrasensitive immunoassay procedures based on the ELISA (enzyme linked immuno absorbent assay) technology were employed for the measurement of VEGF, TNF-alpha, GMCSF, GCSF IL4 and IL6. The assays were performed by the Cytokine Laboratory in the VA Hospital at the University of Maryland Baltimore and data provided as optical density readouts that were converted relative to a standard into pg/ml absolute cytokine values. The IL4 and 6 measurements were all negative; however VEGF, TNF-alpha, GMCSF and GCSF yielded interesting and differential results between tumor and normal stem cells. While cytokine levels decreased after RHPS4 treatment in tumor stem cell assays, they increased in normal stem cell cultures.

Since RHPS4 damages telomeres and inhibits telomeres (by displacement) at once, in one embodiment RHPS4 works in killing e.g. leukemic and other cancer stem cells. However, in another embodiment, the bulk cancer cell mass will need to be eradicated by addition of a standard cytotoxic drug such as etoposide (in leukemia) or cisplatin (in lung cancer), for example. Since these agents damage the bone marrow, the additional induction of proliferation from RHPS4 in normal bone marrow stem cells is beneficial.

The foregoing is considered as illustrative only of the principles of the invention. Further, since numerous modifications and changes will readily occur to those skilled in the art, it is not desired to limit the invention to the exact construction and operation shown and described, and, accordingly, all suitable modifications and equivalents may be resorted to, falling within the scope of the invention.

### REFERENCES

All patents and publications mentioned in the specification are indicative of the level of those skilled in the art to which the invention pertains. All patents and publications cited herein are incorporated by reference to the same extent as if each individual publication was specifically and individually indicated to be incorporated by reference.
U.S. Pat. No. 7,115,619
Alley M C, Hollingshead M G, Dykes D J, Waud W R (2004) Human tumor xenograft models in NCI drug development. Anticancer drug development guide: preclinical screening, clinical trials, and approval. In: Teicher B A, Andrews P A (ed) pp 125-152. Humana Press, Inc. Totowa, N.J.
Armanios M, Greider C W (2005) Telomerase and Cancer Stem Cells. Cold Spring Harbor Symposia on Quantitative Biology, Volume LXX. Cold Spring Harbor Laboratory Press 0-87969-773-3. doi:10.1101/sqb.2005.70.030
Blackburn E H (1991) Structure and function of telomeres. Nature 350: 569-573.
Blackburn E H (2000) Telomere states and cell fates. Nature 408: 53-56.
Blackburn E. H. (2001) Switching and signaling at the telomere. Cell 106: 661-673.
Blanpain C, Lowry W E, Geoghegan A, Polak L, Fuchs E. Self-renewal, multipotency, and the existence of two cell populations within an epithelial stem cell niche. Cell (2004) 118: 635-648.
Blasco M A. (2002) Telomerase beyond telomeres. Nat Rev Cancer 2: 627-633.
Blasco M A (2004) Telomere epigenetics: a higher-order control of telomere length in mammalian cells. Carcinogenesis 25: 1083-1087.
Blasco M A (2005) Telomeres in cancer and aging: lessons from the mouse. Cancer Letters 194: 183-188.
Burger A M (1999) Telomerase in cancer diagnosis and therapy: a clinical perspective. BioDrugs 12:413-422.
Burger A M, Dai F, Schultes C M, Reszka A P, Moore M J, Double J A, Neidle S (2005) The G-quadruplex-interactive molecule BRACO-19 inhibits tumor growth, consistent with telomere targeting and interference with telomerase function. Cancer Res 65:1489-1496.
Burger A. M (2007) Highlights in experimental therapeutics. Cancer Lett. 245: 11-21. doi:10.1016/j.canlet.2006.03.012
Chumsri S, Matsui W, Burger A M. Leukemic stem cell pathways—therapeutic implications. Clin. Cancer Res. 13:6549-54, 2007.
Chumsri S, Phatak P, Edelman M J, Khakpour N, Hamburger A W, Burger A M: Cancer stem cells and individualized therapy. Cancer Genomics Proteomics (2007) 4(3):165-174.
Chumsri S, Burger A M. Cancer stem cells: therapeutic approaches ad consequences targeting stem cells. Current Opin. In Molec. Ther., 2008.
Chou T C, Talalay P (1984) Quantitative analysis of dose-effect relationships: the combined effects of multiple drugs or enzyme inhibitors. Adv Enzyme Regul 22: 27-55.
Clarke M F, Dick J E, Dirks P B, Eaves C J, Jamieson C H, Jones D L, Visvader J, Weissman I L, Wahl G M: Cancer Stem Cells—Perspectives on Current Status and Future Directions: AACR Workshop on Cancer Stem Cells. Cancer Res (2006) 66(19):9339-9344.
Cookson J C, Dai F, Smith V, Heald R A, Laughton C A, Stevens M F, Burger A M (2005) Pharmacodynamics of the G-quadruplex-stabilizing telomerase inhibitor 3,11-difluoro-6,8,13-trimethyl-8H-quino[4,3,2-kl]acridinium methosulfate (RHPS4) in vitro: activity in human tumor cells correlates with telomere length and can be enhanced, or antagonized, with cytotoxic agents. Mol Pharmacol 68:1551-1558.
d'Adda di Fagagna F, Reaper P M, Clay-Farrace L, Fiegler H, Carr P, Von Zglinicki T, Saretzki G, Carter N P, Jackson S P (2003) DNA damage checkpoint response in telomere-initiated senescence. Nature 426: 194-198.
Dikmen Z G, Gellert G C, Jackson S, Gryaznov S, Tressler R, Dogan P, Wright W E, Shay J W (2005) In vivo inhibition of lung cancer by GRN163L: a novel human telomerase inhibitor. Cancer Res 65:7866-7873.
Donnenberg V S, Donnenberg A D (2005) Multiple drug resistance in cancer revisited: the cancer stem cell hypothesis. J Clin Pharmacol 45: 872-877.
Fiebig H H, Burger A M (2001) Human tumor xenografts and explants. Animal models in cancer research. In: Teicher B A (ed) pp 113-137. Humana Press, Inc Totowa, N.J.
Fiebig H H, Maier A, Burger A M (2004) Clonogenic Assay with Established Human Tumor Xenografts: Correlation of In Vitro to In Vivo Activity as a Basis for Anticancer Drug Discovery. Eur J Cancer 40: 802-820.
Fordyce C A, Heaphy C M, Joste N E, et al. Association between cancer-free survival and telomere DNA content in prostate tumors. J Urol. 173: 610-614, 2005.
Geran R I, Greenberg N H, MacDonald M M, Schumacher A M, Abbott B J (1972) Protocols for screening chemical agents and natural products against animal tumors and other biological systems. Cancer Chemother Rep 3:1-103.
Gisselsson D, Jonson T, Petersen A, Strombeck B, Dal Cin P, Hoglund M, Mitelman F, Mertens F, Mandahl N (2001) Telomere dysfunction triggers extensive DNA fragmentation and evolution of complex chromosome abnormalities in human malignant tumors. Proc Natl Acad Sci USA 98: 12683-12688.
Goodell M A, Brose K, Paradis G, Conner A S, Mulligan R C (1996) Isolation and functional properties of murine hematopoietic stem cells that are replicating in vivo. J Exp Med 183: 1797-1806.
Gowan S M, Heald R, Stevens M F, Kelland L R (2001) Potent inhibition of telomerase by small-molecule pentacyclic acridines capable of interacting with G-quadruplexes. Mol Pharmacol 60:981-988.
Gowan S M, Harrison J R, Patterson L, Valenti M, Read M A, Neidle S, Kelland L R (2002) A G-quadruplex-interactive potent small-molecule inhibitor of telomerase exhibiting in vitro and in vivo antitumor activity. Mol Pharmacol 61:1154-1162.
Griffith J D, Comeau L, Rosenfield S, Stansel R M, Bianchi A, Moss H, de Lange T (1999) Mammalian telomeres end in a large duplex loop. Cell 97: 503-514.
Hahn W C, Stewart S A, Brooks M W, York S G, Eaton E, Kurachi A, Beijersbergen R L, Knoll J H, Meyerson M, Weinberg R A (1999) Inhibition of telomerase limits the growth of human cancer cells. Nat Med 5:1164-1170.
Hamburger A W, Salmon S E (1977). Primary bioassay of human tumor stem cells. Science 197:461-463.
Hanahan D, Weinberg R A (2000) The hallmarks of cancer. Cell: 100:57-70.
Hao L Y, Strong M A, Greider C W (2004) Phosphorylation of H2AX at short telomeres in T cells and fibroblasts. J Biol Chem 279: 45148-45154.
Hao L Y, Armanios M, Strong M A, Karim B, Feldser D M, Huso D, Greider C W (2005) Short telomeres, even in the presence of telomerase, limit tissue renewal capacity. Cell 123: 1121-1131.
Hayflick L, Moorhead P S (1961) The serial cultivation of human diploid cell strains. Exp Cell Res 25: 585-621.
Heald R A, Modi C, Cookson J C, Hutchinson I, Laughton C A, Gowan S M, Kelland L R, Stevens M F (2002). Antitumor polycyclic acridines. 8.(1) Synthesis and telomerase-inhibitory activity of methylated pentacyclic acridinium salts. J Med Chem 45 (3)590-7.
Hermann P C, Huber S L, Herrler T, Aicher A, Ellwart J W, Guba M, Bruns C J, Heeschen C. Cell Stem Cell 13; 1(3):313-23 (2007)
Hill R P, Perris R: “Destemming” cancer stem cells. J Natl Cancer Inst (2007) 99(19):1435-1440.
Hirschmann-Jax, C, Foster A E, Wulf G G, et al. A distinct “side population” of cells with high drug efflux capacity in human tumor cells, Proc. Natl. Acad. Sci. 101: 14228-14233, 2004.
Hochreiter A E, Xiao H, Goldblatt E M, Gryaznov S M, Miller K D, Badve S, Sledge G W, Herbert B S (2006) Telomerase template antagonist GRN163L disrupts telomere maintenance, tumor growth, and metastasis of breast cancer. Clin Cancer Res 12:3184-3192.
Holt S E, Shay J W, Wright W E (1996) Refining the telomere-telomerase hypothesis of aging and cancer. Nature Biotechnol 14: 1734-1741.
IJpma A S, Greider C W (2003) Short Telomeres Induce a DNA Damage Response in Saccharomyces cerevisiae Mol Biol Cell 14: 987-1001.
Ju Z, Rudolph K L (2006) Telomeres and telomerase in cancer stem cells. Eur J Cancer 42:1197-1203.
Incles C M, Schultes C M, Kempski H., Koehler H, Kelland L R, Neidle S (2004) A G-quadruplex telomere targeting agent produces p16-associated senescence and chromosomal fusions in human prostate cancer cells. Mol Cancer Ther 3: 1201-1206.
Kelland L R (2005) Overcoming the immortality of tumor cells by telomere and telomerase based cancer therapeutics—current status and future prospects. Eur J Cancer 41: 971-979.
Kim N R, Piatysek M A, Prowse K R, Harley C B West M D, Ho P L C, Corielo G M, Wright W E, Weinrich S L, Shay J W (1994) Specific association of human telomerase activity with immortal cells and cancer Science 266: 2011-2015.
Kim et al., J. Am. Chem. Soc., 124: 2098-2099, 2002.
Leonetti C, Amodei S, D'Angelo C, Rizzo A, Benassi B, Antonelli A, Elli R, Stevens M F, D'Incalci M, Zupi G, Biroccio A (2004) Biological activity of the G-quadruplex ligand RHPS4 (3,11-difluoro-6,8,13-trimethyl-8H-quino[4,3,2-kl]acridinium methosulfate) is associated with telomere capping alteration. Mol Pharmacol 66: 1138-1146.
Locke M, Heywood M, Fawell S, Mackenzie I C (2005) Retention of intrinsic stem cell hierarchies in carcinoma-derived cell lines. Cancer Res 65:8944-8950.
Makarov V L, Hirose Y, Langmore J P (1997) Long G tails at both ends of human chromosomes suggest a C strand degradation mechanism for telomere shortening. Cell 88: 657-666.
Masutomi K, Yu E Y, Khurts S, Ben-Porath I, Currier J L, Metz G B, Brooks M W, Kaneko S, Murakami S, DeCaprio J A, Weinberg R A, Stewart S A, Hahn W C (2003) Telomerase maintains telomere structure in normal human cells. Cell 114: 241-253.
Meng L, Kohlhagen G, Liao Z, Antony S, Sausville E, Pommier E (2005) DNA-Protein Cross-links and Replication-Dependent Histone H2AX Phosphorylation Induced by Aminoflavone (NSC 686288), a Novel Anticancer Agent Active against Human Breast Cancer Cells Cancer Research 65: 5337-5343.
Mosmann T (1983) Rapid colorimetric assay for cellular growth and survival: application to proliferation and cytotoxicity assays. J Immunol Methods 65: 55-63.
Ogawa M, Parmely R T, Bank H L, et al. Human marrow erythropoiesis in culture. I. Characterization of methylcellulose colony assay. Blood 48:407-417, 1976.
Parkinson G N, Lee M P H, Neidle S (2002) Crystal structure of parallel quadruplexes from human telomeric DNA. Nature 417: 876-880.
Phatak P, Burger A M. Telomerase and its potential for therapeutic intervention. Br. J. Pharmacol. 152:1003-11, 2007.
Phatack P, Cookson J C, Dai F, Smith V, Gartenhaus R B, Stevens M F G, Burger A M. Telomere uncapping by the G-quadruplex ligand RHPS4 inhibits clonogenic tumor cell growth in vitro and in vivo consistent with a cancer stem cell targeting mechanism. Br. J. Pharmacol. 96:1223-33, 2007.
Phatak P, Cookson J C, Dai F, Smith V, Gartenhaus R B, Stevens M F G, and Burger A M. British Journal of Cancer 96, 1223-1233 (2007)
Reed J E, Arnal A A, Neidle S, Vilar R (2006) Stabilization of G-Quadruplex DNA and Inhibition of Telomerase Activity by Square-Planar Nickel(II) Complexes. J Am Chem Soc 128: 5992-5993.
Remington's Pharmaceutical Sciences, 18th Ed. Mack Printing Company, 1990.
Riou et al., PNAS 99(5): 2672-2677 (2002)
Salvati E, Leonetti C, Rizzo A, Scarsella M, Mottolese M, Galati R, Sperduti I, Stevens M, D'Incalci M, Blasco M, Chiorino G, Horard B, Gilson E, Stoppacciaro A, Zupi G, Biroccio A (2007) Telomere damage promotes antitumoral activity of the G-quadruplex ligand RHPS4. J Clinc Invest, in press.
Sarin K Y, Cheung P, Gilison D, Lee E, Tennen R I, Wang E, Artandi M K, Oro A E, Artandi S E (2005) Conditional telomerase induction causes proliferation of hair follicle stem cells. Nature 436: 1048-1052.
Shammas et al., Mol Cancer Ther. 2:825-833, 2003.
Shammas et al., Clinical Cancer Research Vol. 10, 770-776, 2004.
Shammas et al., Gastroenterology 126(5):1337-46, 2004.
Smith C D, Blackburn E H (1999) Uncapping and deregulation of telomeres lead to detrimental cellular consequences in yeast. J Cell Biol 145: 203-214.
Sung Y H, Choi Y S, Cheong C, Lee H W (2005) The pleiotropy of telomerase against cell death. Mol Cell 19: 303-309.
Tahara H, Shin-Ya K, Seimiya H, Yamada H, Tsuruo T, Ide T (2006) G-Quadruplex stabilization by telomestatin induces TRF2 protein dissociation from telomeres and anaphase bridge formation accompanied by loss of the 3′ telomeric overhang in cancer cells. Oncogene 25:1955-66.
Wang J C Y, Warner J K, Erdmann N, Lansdorp P M, Harrington L, Dick J E (2005) Dissociation of telomerase activity and telomere length maintenance in primitive human hematopoietic cells. Proc Natl Acad Sci USA 102: 14398-14403, www.pnas.org_cgi_doi_{—}10.1073_pnas.0504161102
Williamson J R (1994) G-quartet structures in telomeric DNA. Annu Rev Biophys Biomol Struct 23:703-730.
Workman P, Twentyman P, Balkwill F et al. (1998) United kingdom co-ordinating committee on cancer research (UKCCCR) guidelines for the welfare of animals in experimental neoplasia (2nd ed). Br J Cancer 77:1-10.
Zahler A M, Williamson J R, Cech W R, Prescott D M (1991) Inhibition of telomerase by G-quartet structures Nature 350: 718-720.
Zhou S, Schuetz J D, Bunting K D, et al. The ABC transporter Bcrp1/ABCG2 is expressed in a wide variety of stem cells and is a molecular determinant of the side-population phenotype, Nat. Med. 7: 1028-1034, 2001.

What is claimed is:

1. A method of inhibiting the growth of a cancer stem cell, comprising contacting said cancer stem cell with an effective amount of G-quadruplex ligand.

2. The method of claim 1, wherein the G-quadruplex ligand comprises 3,11-difluoro-6,8,13-trimethyl-8H-quino[4,3,2-kl]acridinium methosulfate (RHPS4), BRACO-19, telomestatin, or a functionally active derivative thereof.

3. The method of claim 2, wherein the G-quadruplex ligand comprises 3,11-difluoro-6,8,13-trimethyl-8H-quino[4,3,2-kl]acridinium methosulfate (RHPS4).

4. The method of claim 1, further comprising contacting said cancer stem cell with an additional anti-cancer drug.

5. The method of claim 4, wherein the additional anti-cancer drug acts additively or synergistically with the G-quadruplex ligand.

6. The method of claim 4, wherein the additional anti-cancer drug is selected from the group consisting of a mitotic spindle poison, a heat shock protein inhibitor, an anti-metabolite, a cross-linking agent, a platinum compound, an arsenical, a HDAC inhibitor, a Poly(ADP-Ribose) polymerase (PARP) inhibitor, hTERT transcription inhibitor, a dyskerin antisense compound, gemcitabine, and a double strand break (DSB)-inducing agent.

7. The method of claim 7, wherein the mitotic spindle poison comprises Paclitaxel, Vincristine, Vinblastine, Vinorelbine, an aurora kinase inhibitor, Vinflunine, docetaxel, or an epithiolone.

8. The method of claim 7, wherein the hTERT transcription inhibitor is sodium metaarsenite, GRN163L, or arsenic trioxide.

9. The method of claim 7, wherein the heat shock protein inhibitor comprises 17-AAG, 17-DMAG, CNF1010, or IPI-504.

10. The method of claim 7, wherein the DSB-inducing agent comprises doxorubicin, topotecan, irinotecan, oxaliplatin, cyclophosphamide, temozolomide, daunorubicin, or epirubicin.

11. The method of claim 7, wherein the platinum compound comprises cisplatin or carboplatin.

12. The method of claim 1, wherein said method further comprises a step of determining the presence of a cancer stem cell.

13. The method of claim 12, said step comprising assaying for the presence of one or more specific cell surface markers that are present on cancer stem cells.

14. A method of treating cancer in a mammal in need of such treatment by inhibiting the growth of a cancer stem cell, comprising administering a therapeutically effective amount of a G-quadruplex ligand to the mammal.

15. The method of claim 14, wherein the G-quadruplex ligand comprises 3,11-difluoro-6,8,13-trimethyl-8H-quino[4,3,2-kl]acridinium methosulfate (RHPS4), BRACO-19, telomestatin, or a functionally active derivative thereof.

16. The method of claim 15, wherein the G-quadruplex ligand comprises 3,11-difluoro-6,8,13-trimethyl-8H-quino[4,3,2-kl]acridinium methosulfate (RHPS4).

17. The method of claim 14, further comprising contacting said cancer stem cell with an additional anti-cancer drug.

18. The method of claim 17, wherein the additional anti-cancer drug acts additively or synergistically with the G-quadruplex ligand.

19. The method of claim 17, wherein the additional anti-cancer drug is selected from the group consisting of a mitotic spindle poison, a heat shock protein inhibitor, an anti-metabolite, a cross-linking agent, a platinum compound, an arsenical, a HDAC inhibitor, a Poly(ADP-Ribose) polymerase (PARP) inhibitor, an inhibitor of hTERT transcription, dyskerin antisense compound, and a double strand break (DSB)-inducing agent, or gemcitabine.

20. The method of claim 19, wherein the mitotic spindle poison comprises Paclitaxel, Vincristine, Vinblastine, Vinorelbine, an aurora kinase inhibitor, Vinflunine, docetaxel, or an epithiolone.

21. The method of claim 19, wherein the hTERT transcription inhibitor is sodium metaarsenite, GRN163L, or arsenic trioxide.

22. The method of claim 19, wherein the heat shock protein inhibitor comprises 17-AAG, 17-DMAG, CNF1010, or IPI-504.

23. The method of claim 19 wherein the DSB-inducing agent comprises doxorubicin, topotecan, irinotecan, oxaliplatin, cyclophosphamide, temozolomide, daunorubicin, or epirubicin.

24. The method of claim 19, wherein the platinum compound comprises cisplatin or carboplatin.

25. The method of claim 14, wherein said method further comprises a step of determining the presence of a cancer stem cell.

26. The method of claim 25, said step comprising assaying for the presence of one or more specific cell surface markers that are present on cancer stem cells.

27. The method of claim 14, wherein the therapeutically effective amount of the G-quadruplex ligand causes non-cancerous cell proliferation in the mammal.

28. The method of claim 27, wherein the non-cancerous cell is a non-cancerous stem cell selected from the group consisting of a normal stem cell, an adult stem cell, or an embryonic stem cell.

29. A method of treating cancer in a mammal in need of such treatment comprising administering a therapeutically effective amount of a G-quadruplex ligand to the mammal, wherein the G-quadruplex ligand induces non-cancerous cell proliferation and inhibits the growth of cancer cells.

30. The method of claim 29, wherein the cancer cells are cancer stem cells.

31. The method of claim 29, wherein the non-cancerous cells are non-cancerous stem cells.

32. The method of claim 29, wherein the G-quadruplex ligand comprises 3,11-difluoro-6,8,13-trimethyl-8H-quino[4,3,2-kl]acridinium methosulfate (RHPS4).

33. The method of claim 32, wherein the concentration of the therapeutically effective amount of G-quadruplex ligand is between 0.01 micromolar and 1 micromolar.

34. A method of increasing proliferation of a non-cancerous stem cell in an individual, comprising the step of delivering an effective amount of a G-quadruplex ligand to the individual.

35. The method of claim 34, wherein the method increases proliferation of a non-cancerous stem cell in an individual.

36. The method of claim 34, wherein the method increases proliferation of a non-cancerous stem cell in vitro.
